(19) United States
(12) Patent Application Publication
(10) Pub. No.: US 2023/0280357 A1
(43) Pub. Date: Sep. 7, 2023

(54) CSF PHOSPHORYLATED TAU AND AMYLOID BETA PROFILES AS BIOMARKERS OF TAUOPATHIES

(71) Applicant: Washington University, St. Louis, MO (US)

(72) Inventors: CHIHIRO SATO, St. Louis, MO (US); NICOLAS BARTHELEMY, St. Louis, MO (US); RANDALL BATEMAN, St. Louis, MO (US)

(21) Appl. No.: 17/798,514

(22) PCT Filed: Mar. 31, 2022
(86) PCT No.: PCT/US2022/022906

§ 371 Date: Aug. 9, 2022

Related U.S. Application Data
(60) Provisional application No. 63/169,193, filed on Mar. 31, 2021.

(51) Int. Cl. G01N 33/68

(52) U.S. Cl. CPC G01N 33/6896; G01N 2333/4709; G01N 2440/14; G01N 2800/2821

(57) ABSTRACT

The present disclosure provides methods to quantify tau phosphorylation at specific amino acid residues and optionally Ab species to diagnose a subject, guide treatment decisions, and select subjects for clinical trials.

### CROSS REFERENCE TO RELATED APPLICATIONS

This application claims the priority of U.S. Provisional Application No. 63/169,193, filed Mar. 31, 2021, the disclosure of which is hereby incorporated by reference in its entirety.

### FIELD

The present disclosure encompasses the use of tau and amyloid-beta species in CSF to measure pathological features and/or clinical symptoms of 3Rand 4R- tauopathies in order to diagnose and/or choose treatments appropriate for a given disease.

### BACKGROUND

The microtubule-associated protein tau (MAPT or tau) plays an essential role in the morphology and physiology of neurons. Tau has six different isoforms of the full-length protein and undergoes a number of possible post-translational modifications including acetylation, glycosylation and phosphorylation. Phosphorylation is important for regulating the normal function of tau in axonal stabilization and can occur at over 80 different residues. However, excessive phosphorylation of tau appears to increase the probability of tau aggregating into intracellular insoluble paired helical filaments (PHF) and neurofibrillary tangles (NFT), which are primarily composed of hyperphosphorylated tau.

Intracellular neurofibrillary tangles in the cerebral cortex are a defining pathological feature of Alzheimer disease (AD) and correlate with the onset of clinical symptoms long after the appearance of extracellular amyloid-β (Aβ) plaques, which begin to develop up two decades before symptom onset. In AD, soluble p-tau and unphosphorylated tau are increased by two-fold in the cerebrospinal fluid (CSF). It has been proposed that these changes reflect the effects of neuronal death (neurodegeneration) passively releasing tau and NFT into the CSF. However, in other tauopathies with significant NFT pathology and neurodegeneration (e.g. progressive supranuclear palsy, frontotemporal lobar degeneration-tau), CSF levels of soluble p-tau and total tau do not increase.

Other tauopathies or neurodegenerative diseases such as Progressive Supranuclear Palsy (PSP), Corticobasal Syndrome (CBS), and Frontotemporal Dementia (FTD), currently have no CSF or imaging biomarkers and diagnosis primarily depends on clinical assessment ultimately confirmed after brain autopsy. It remains unclear whether other tauopathies induce p-tau modifications in the absence of amyloid pathology.

Accordingly, there remains a need in the art for improved methods for the reliable and accurate clinical diagnoses of tauopathies, with implications for the design and implementation of clinical trials.

### SUMMARY

Among the various aspects of the present disclosure are provided methods to quantify tau phosphorylation at specific amino acid residues and optionally quantify Aβ species to diagnose a subject, guide treatment decisions, and select subjects for clinical trials.

One aspect of the present disclosure encompasses a method of discriminating a MAPT R406W tauopathy, the method generally comprising (a) providing a processed CSF or blood sample obtained from a subject, wherein the CSF or blood sample is enriched for one or more ptau and Aβ species; and (b) quantifying, in the processed sample, a pT217/T217 value and an Aβ 42/40 value, wherein an increase in the pT217/T217 value and a normal Aβ 42/40 value discriminates a MAPT R406W tauopathy from a healthy state.

Another aspect of the present disclosure encompasses a method of discriminating a MAPT R406W tauopathy, the method generally comprising (a) providing a processed CSF or blood sample obtained from a subject, wherein the CSF or blood sample is enriched for one or more ptau and Aβ species; and (b) quantifying, in the processed sample, pT217/T217 value and Aβ 42/40 value, wherein a decrease in the pT217/T217 value and an increase Aβ 42/40 value discriminates a MAPT R406W tauopathy from Alzheimer’s disease.

Another aspect of the present disclosure encompasses a method of discriminating a MAPT R406W tauopathy, the method generally comprising (a) providing a processed CSF or blood sample obtained from a subject, wherein the CSF or blood sample is enriched for one or more ptau and Aβ species; and (b) quantifying, in the processed sample, a composite pT217/T217 × Aβ 42/40 value, wherein the pT217/T217 × Aβ 42/40 value discriminates a MAPT R406W tauopathy from Alzheimer’s disease, 4R-tauopathy and a healthy state. In some embodiments, an increased pT217/T217 × Aβ 42/40 value quantified in step (b) relative to a pT217/T217 × Aβ 42/40 value in a healthy control population discriminates a MAPT R406W tauopathy from a healthy state. In some embodiments, an increased pT217/T217 × Ab 42/40 value quantified in step (b) relative to a pT217/T217 × Aβ 42/40 value in an Alzheimer’s disease population discriminates a MAPT R406W tauopathy from AD. In some embodiments, an increased pT217/T217 × Aβ 42/40 value quantified in step (b) relative to a pT217/T217 × Aβ 42/40 value in a 4R-tauopathy population discriminates a MAPT R406W tauopathy from a 4R-tauopathy.

Another aspect of the present disclosure encompasses a method of discriminating a sporadic frontotemporal dementia (FTD), the method generally comprising (a) providing a processed CSF or blood sample obtained from a subject, wherein the CSF or blood sample is enriched for one or more ptau species; and (b) quantifying, in the processed sample, a pT181/T181 value, wherein the pT181/T181 value discriminates a sporadic FTD from Alzheimer’s disease, non-sporadic FTD tauopathies and a healthy state. In some a decreased pT181/T181 value quantified in step (b) relative to a pT181/T181 value in an Alzheimer’s disease population discriminates a sporadic FTD from Alzheimer’s disease. In some embodiments, a decreased pT181/T181 value quantified in step (b) relative to a pT1811T181 value in a healthy control population discriminates a sporadic FTD from a healthy state. In some embodiment, a decreased pT181/T181 value quantified in step (b) relative to a pT181/T181 value in a non-sporadic FTD tauopathy population discriminates a sporadic FTD from a non-sporadic FTD tauopathy.

Another aspect of the present disclosure encompasses a method to diagnose a subject having a symptom of Alzheimer’s disease, the method generally comprises (a) providing a processed CSF or blood sample obtained from the subject, wherein the CSF or blood sample is enriched for one or more ptau and Aβ species and quantifying, in the processed sample, a pT217/T217 value and a Aβ 42/40 value; and (b) diagnosing the subject as having Frontotemporal Dementia (FTD) or at an increased risk of FTD when an increase in the pT217/T217 value and a normal Aβ 42/40 value are detected relative to a pT217/T217 value and an Aβ 42/40 value in a healthy control population. In some embodiments, the method further comprises determining a composite pT217/T217 × Aβ 42/40 value, wherein an increased composite value relative to a healthy control indicates the subject as having FTD or at an increased risk for FTD. In some embodiments, the subject is diagnosed as a MAPT R406W mutation carrier.

Another aspect of the present disclosure encompasses a method to diagnose a subject having a symptom of Alzheimer’s disease, the method generally comprises (a) providing a processed CSF or blood sample obtained from the subject, wherein the CSF or blood sample is enriched for one or more ptau and Aβ species and quantifying, in the processed sample, a pT217/T217 value and a Aβ 42/40 value; and (b) diagnosing the subject as having Frontotemporal Dementia (FTD) or at an increased risk of FTD when an decrease in the pT217/T217 value and an increase in the Aβ 42/40 value are detected relative a pT217/T217 value and a Aβ 42/40 value in an Alzheimer’s disease population. In some embodiments, the method further comprising determining a composite pT217/T217 × Aβ 42/40 value, wherein an increased composite value relative to a Alzheimer’s disease population indicates the subject as having FTD or at an increased risk for FTD. In some embodiments, the subject is diagnosed as a MAPT R406W mutation carrier.

Another aspect of the present disclosure encompasses a method to diagnose a subject having a symptom of Alzheimer’s disease, the method generally comprises (a) providing a processed CSF or blood sample obtained from the subject, wherein the CSF or blood sample is enriched for one or more ptau species and quantifying, in the processed sample, a pT181/T181 value; and (b) diagnosing the subject as having sporadic Frontotemporal Dementia (FTD) or at an increased risk of sporadic FTD when a decrease in the pT181/T181 value are detected relative to a pT181/T181 value in a healthy control population.

Another aspect of the present disclosure encompasses a method to diagnose a subject having a symptom of Alzheimer’s disease, the method generally comprises (a) providing a processed CSF or blood sample obtained from the subject, wherein the CSF or blood sample is enriched for one or more ptau species and quantifying, in the processed sample, a pT181/T181 value; and (b) diagnosing the subject as having sporadic Frontotemporal Dementia (FTD) or at an increased risk of sporadic FTD when a decrease in the pT181/T181 value are detected relative to a pT181/T181 value in an Alzheimer’s disease population.

Another aspect of the present disclosure encompasses a method for measuring MAPT R406W tauopathy disease progression in a subject, the method generally comprises (a) providing a first processed CSF or blood sample obtained from a subject, wherein the first CSF or blood sample is enriched for one or more ptau and Aβ species and quantifying, in the first processed sample, pT217/T217 value, and Aβ 42/40 value; (b) providing a second processed CSF or blood sample obtained from the subject after the first sample, wherein the second CSF or blood sample is enriched for the same ptau and Aβ species as in step (a) and quantifying, in the second processed sample, a pT217/T217 value and Aβ 42/40 value; and (c) calculating the difference between the quantified pT217/T217 value and Aβ 42/40 value in the second sample and the first sample, wherein a statistically significant difference in the quantified pT217/T217 value, and Aβ 42/40 value in the second sample indicates progression of the subject’s disease.

Another aspect of the present disclosure encompasses a method for measuring MAPT R406W tauopathy disease progression in a subject, the method generally comprises (a) providing a first processed CSF or blood sample obtained from a subject, wherein the first CSF or blood sample is enriched for one or more ptau and Aβ species and quantifying, in the first processed sample, a composite pT217/T217 × Aβ 42/40 value; (b) providing a second processed CSF or blood sample obtained from the subject after the first sample, wherein the second CSF or blood sample is enriched for the same ptau and Aβ species as in step (a) and quantifying, in the second processed sample, a composite pT217/T217 × Ab 42/40 value; and (c) calculating the difference between the quantified composite pT217/T217 × Aβ 42/40 value in the second sample and the first sample, wherein a statistically significant difference in the quantified composite pT217/T217 × Aβ 42/40 value in the second sample indicates progression of the subject’s disease.

Another aspect of the present disclosure encompasses a method for measuring sporadic frontotemporal dementia (FTD) disease progression in a subject, the method generally comprises (a) providing a first processed CSF or blood sample obtained from a subject, wherein the first CSF or blood sample is enriched for one or more ptau and quantifying, in the first processed sample, a pT181/T181 value; (b) providing a second processed CSF or blood sample obtained from the subject after the first sample, wherein the second CSF or blood sample is enriched for the same ptau species as in step (a) and quantifying, in the second processed sample, a pT181/T181 value; and (c) calculating the difference between the quantified a pT181/T181 value in the second sample and the first sample, wherein a statistically significant difference in the quantified a pT1811T181 value in the second sample indicates progression of the subject’s disease.

Another aspect of the present disclosure encompasses a method for treating a subject in need thereof, the method generally comprises (a) providing a processed CSF or blood sample obtained from a subject, wherein the CSF or blood sample is enriched for one or more ptau and Aβ species and quantifying, in the processed sample, a pT217/T217 value and an Aβ 42/40 value; and (b) administering a pharmaceutical composition to the subject when an increase in the pT217/T217 value and a normal Aβ 42/40 value is detected relative to a pT217/T217 value and an Aβ 42/40 value in a healthy control population. In some embodiments, the pharmaceutical composition comprises a FTD therapy.

Another aspect of the present disclosure encompasses a method for treating a subject in need thereof, the method generally comprises (a) providing a processed CSF or blood sample obtained from a subject, wherein the CSF or blood sample is enriched for one or more ptau and Aβ species and quantifying, in the processed sample, a pT217/T217 value and an Aβ 42/40 value; and (b) administering a pharmaceutical composition to the subject when a decrease in the pT217/T217 value and an increase Aβ 42/40 value is detected relative pT217/T217 value and an Aβ 42/40 value in an Alzheimer’s disease population. In some embodiments, the pharmaceutical composition comprises a FTD therapy.

Another aspect of the present disclosure encompasses a method for treating a subject in need thereof, the method generally comprises (a) providing a processed CSF or blood sample obtained from a subject, wherein the CSF or blood sample is enriched for one or more ptau and Aβ species and quantifying, in the processed sample, a composite pT217/T217 × Aβ 42/40 value; and (b) administering a pharmaceutical composition to the subject when an increase in the composite pT217/T217 × Aβ 42/40 value is detected relative composite pT217/T217 × Aβ 42/40 value in an healthy control population, in an Alzheimer’s disease population or in a 4R-tauopathy population. In some embodiment, the pharmaceutical composition comprises a FTD therapy.

Another aspect of the present disclosure encompasses a method for treating a subject in need thereof, the method generally comprises (a) providing a processed CSF or blood sample obtained from a subject, wherein the CSF or blood sample is enriched for one or more ptau and quantifying, in the processed sample, a pT181/T181 value; and (b) administering a pharmaceutical composition to the subject when a decrease in the pT181/T181 value is detected relative to a pT181/T181 value in an healthy control population, in an Alzheimer’s disease population or in a non-sporadic FTD tauopathy population. In some embodiments, the pharmaceutical composition comprises a sporadic FTD therapy.

In each of the above aspects, in certain embodiments, the treatment does not include a therapeutic agent to reduce or prevent Aβ deposition and/or the treatment alters or stabilizes the amount of the quantified ptau species.

Another aspect of the present disclosure encompasses a method for selecting a subject in a clinical trial, the method generally comprises (a) providing a processed CSF or blood sample obtained from a subject, wherein the CSF or blood sample is enriched for one or more ptau and Aβ species; (b) quantifying, in the processed sample, pT217/T217 value and Aβ 42/40 value; and (c) selecting the subject into a clinical trial for FTD when pT217/T217 value is increased and Aβ 42/40 value is about the same as a healthy control population.

Another aspect of the present disclosure encompasses a method for selecting a subject in a clinical trial, the method generally comprises (a) providing a processed CSF or blood sample obtained from a subject, wherein the CSF or blood sample is enriched for one or more ptau and Aβ species; (b) quantifying, in the processed sample, pT217/T217 value and Aβ 42/40 value; and (c) excluding the subject into a clinical trial for AD or Ab therapy when pT217/T217 value is increased and Aβ 42/40 value is about the same as a healthy control population.

Another aspect of the present disclosure encompasses a method for selecting a subject in a clinical trial, the method generally comprises (a) providing a processed CSF or blood sample obtained from a subject, wherein the CSF or blood sample is enriched for one or more ptau and Aβ species; (b) quantifying, in the processed sample, a composite pT217/T217 × Aβ 42/40 value; and (c) selecting the subject into a clinical trial for FTD when the composite pT217/T217 × Aβ 42/40 value is increased relative to a healthy control population.

Another aspect of the present disclosure encompasses a method for selecting a subject in a clinical trial, the method generally comprises (a) providing a processed CSF or blood sample obtained from a subject, wherein the CSF or blood sample is enriched for one or more ptau and Aβ species; (b) quantifying, in the processed sample, a composite pT217/T217 × Aβ 42/40 value; and (c) excluding the subject into a clinical trial for AD when the composite pT217/T217 × Aβ 42/40 value is increased relative to an AD population.

Another aspect of the present disclosure encompasses a method for selecting a subject in a clinical trial, the method generally comprises (a) providing a processed CSF or blood sample obtained from a subject, wherein the CSF or blood sample is enriched for one or more ptau species; (b) quantifying, in the processed sample, a pT181/T181 value; and (c) selecting the subject into a clinical trial for a sporadic FTD therapy when the pT181/T181 value is decreased relative to a healthy control population.

Another aspect of the present disclosure encompasses a method for selecting a subject in a clinical trial, the method generally comprises (a) providing a processed CSF or blood sample obtained from a subject, wherein the CSF or blood sample is enriched for one or more ptau species; (b) quantifying, in the processed sample, a pT181/T181 value; and (c) excluding the subject into a clinical trial for AD when the composite pT181/T181 value is decreased relative to an AD population.

Another aspect of the present disclosure encompasses a method of discriminating a Alzheimer’s disease, the method generally comprises (a) providing a processed CSF or blood sample obtained from a subject, wherein the CSF or blood sample is enriched for one or more ptau species; and (b) quantifying, in the processed sample, a pT153/T153 value, wherein the pT153/T153 value discriminates Alzheimer’s disease from a non-AD tauopathy and a healthy state. In some embodiments, an increased pT153/T153 value relative to a healthy control population and a non-Alzheimer’s disease tauopathy population discriminates from Alzheimer’s disease from a non-AD tauopathy and a healthy state.

Another aspect of the present disclosure encompasses a method for selecting a subject into a clinical trial, the method generally comprises (a) providing a processed CSF or blood sample obtained from a subject, wherein the CSF or blood sample is enriched for one or more ptau species; (b) quantifying, in the processed sample, a pT153/T153 value; and (c) selecting the subject into a clinical trial for an AD therapy when the pT153/T153 value is increased relative to a healthy control population or a non-AD tauopathy population.

Another aspect of the present disclosure encompasses a method for selecting a subject into a clinical trial, the method generally comprised (a) providing a processed CSF or blood sample obtained from a subject, wherein the CSF or blood sample is enriched for one or more ptau species; (b) quantifying, in the processed sample, a pT151/T151 value; and (c) excluding the subject into a clinical trial for a non-AD tauopathy therapy when the pT153/T153 value is increased relative to an non-AD tauopathy population.

Another aspect of the present disclosure encompasses a method of discriminating a Alzheimer’s disease, the method generally comprises (a) providing a processed CSF or blood sample obtained from a subject, wherein the CSF or blood sample is enriched for one or more ptau species; and (b) quantifying, in the processed sample, a pT111/T111 value, wherein the pT111/T111 value discriminates Alzheimer’s disease from a non-AD tauopathy and a healthy state. In some embodiments, an increased pT111/T111 value relative to a healthy control population and a non-Alzheimer’s disease tauopathy population discriminates from Alzheimer’s disease from a non-AD tauopathy and a healthy state.

Another aspect of the present disclosure encompasses a method for selecting a subject into a clinical trial, the method generally comprises (a) providing a processed CSF or blood sample obtained from a subject, wherein the CSF or blood sample is enriched for one or more ptau species; (b) quantifying, in the processed sample, a pT111/T111 value; and (c) selecting the subject into a clinical trial for an AD therapy when the pT111/T111 value is increased relative to a healthy control population or a non-AD tauopathy population.

Another aspect of the present disclosure encompasses a method for selecting a subject into a clinical trial, the method generally comprises (a) providing a processed CSF or blood sample obtained from a subject, wherein the CSF or blood sample is enriched for one or more ptau species; (b) quantifying, in the processed sample, a pT111/T111 value; and (c) excluding the subject into a clinical trial for a non-AD tauopathy therapy when the pT111/T111 value is increased relative to an non-AD tauopathy population.

Another aspect of the present disclosure encompasses a method of discriminating a tauopathy, the method generally comprises (a) providing a processed CSF or blood sample obtained from a subject, wherein the CSF or blood sample is enriched for one or more ptau species; and (b) quantifying, in the processed sample, a pT205/T205 value, wherein the pT205/T205 value discriminates a tauopathy and a healthy state. In some embodiments, an increase pT205/T205 value discriminates a tauopathy and a healthy state.

Another aspect of the present disclosure encompasses a method of discriminating a tauopathy, the method comprising (a) providing a processed CSF or blood sample obtained from a subject, wherein the CSF or blood sample is enriched for one or more ptau species; and (b)quantifying, in the processed sample, a pT208/T208 value, wherein the pT208/T208 value discriminates a tauopathy and a healthy state. In some embodiments, an increase pT208/T208 value discriminates a tauopathy and a healthy state.

These and other aspects and iterations of the invention are described more thoroughly below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The application file contains at least one photograph executed in color. Copies of this patent application publication with color photographs will be provided by the Office upon request and payment of the necessary fee.

FIG. 1A, FIG. 1B, FIG. 1C, FIG. 1D, FIG. 1E and FIG. 1F show MAPT R406W carriers have increased CSF pT217/T217 without Amyloid pathology. FIG. 1A shows all samples (n = 252) were plotted to quadrant analyses using the cutoffs calculated in the WashU-A cohort (CSF Aβ 42/40 cutoff = 0.086, CSF pT217/T217 cutoff = 4.76). FIG. 1B-FIG. 1E show pie charts showing the number of participants in each clinically classified group for each quadrant: II (FIG. 1B), I (FIG. 1C), III (FIG. 1D), and IV (FIG. 1E). CSF, cerebrospinal fluid. FIG. 1F shows a flowchart of cohort analyzed in this study. WashU-A cohort includes AD and age matched controls with a subset (n=48) having PiB PET imaging data. This subset and a PiB PET cutoff of 0.18 were used to determine amyloid positivity of immunoprecipitation and Mass spectrometry (IP/MS) measures of CSF Ab 42/40 (cutoff 0.086). Next, all CSF from WashU-A was used to determine IP/MS CSF pT217/T217 cutoff of 4.76. Cutoff of CSF concentrations of pT181, pT217, total tau, and ratio of pT181/T181 were 44.79, 222.4, 754.7, and 15.75, respectively. WashU-A (n=85), WashU-B (n=88), and Montpellier cohort (n=79) were analyzed for IP/MS CSF Ab 42/40 and pT217/T217 in quadrant analyses (total n=252). Samples were categorized into quadrant I-IV using the cutoffs for amyloid and ptau positivity determined. AMC: age matched control, YNC: Young Normal Control, CBS: Corticobasal Syndrome, PSP: Progressive Supranuclear Palsy, bvFTD: behavioral variant Frontotemporal Dementia.

FIG. 2A, FIG. 2B, FIG. 2C and FIG. 2D show CSF Aβ 42/40, CSF pT217/T217, and composite biomarkers for diagnosis of AD, and MAPT R406W mutation carriers. FIG. 2A shows CSF Aβ 42/40 is significantly decreased in AD (ANOVA, p < 0.0001). FIG. 2B shows CSF pT217/T217 is significantly increased in AD (ANOVA, p < 0.0001). FIG. 2C shows CSF pT217/T217 × CSF Aβ 42/40 is significantly increased in AD compared to Control and 4R tauopathy group consisting mostly of 4R tauopathy (PSP, CBS, FTD-MAPT P301L) and a subset of sporadic bvFTD (may contain FTD-TDP, FTD-FUS and 3R tauopathy. ANOVA, p < 0.0001). This is also significantly increased in MAPT R406W carriers compared to control (ANOVA, p < 0.0001) and 4R tauopathy group (ANOVA, p = 0.0001). FIG. 2D shows CSF pT217/T217 divided by CSF Aβ 42/40, is significantly increased in AD (ANOVA, p < 0.0001). CSF, cerebrospinal fluid; AD, Alzheimer’s disease; PSP, progressive supranuclear palsy; CBS, corticobasal syndrome; bvFTD, behavioral variant frontotemporal dementia.

FIG. 3A, FIG. 3B, FIG. 3C, FIG. 3D, FIG. 3E, FIG. 3F, FIG. 3G, FIG. 3H, FIG. 3I, FIG. 3J, FIG. 3K, and FIG. 3L show ROC analyses of IP/MS CSF Aβ 42/40, CSF tau and ptau. FIG. 3A shows CSF Aβ 42/40 ratio is significantly increased in amyloid PET-PiB+ participants in Wash-A cohort (n=48). FIG. 3B shows CSF Aβ 42/40 can differentiate amyloid PET-PiB+ from PiB- individuals (AUC = 0.9207, p<0.0001). (FIG. 3C, FIG. 3E, FIG. 3G, FIG. 3I, FIG. 3K) CSF pT217/T217 ratio, CSF pT217, CSF T181, CSF total tau concentrations, CSF pT181/T181 ratio are significantly increased in amyloid+ participants defined by CSF Aβ 42/40 ratio in Wash-A cohort (n=85). (FIG. 3D, FIG. 3F, FIG. 3H, FIG. 3J, FIG. 3L) CSF pT217/T217 ratio, CSF pT217, CSF T181, CSF total tau concentrations, CSF pT181/T181 ratio can differentiate CSF Aβ 42/40 positive (amyloid +) from CSF Aβ 42/40 negative (amyloid -) individuals with AUC = 0.983, 0.949, 0.816, 0.693, 0.934, respectively. *p<0.05, **p<0.01, ***p<0.001, ****p<0.0001.

FIG. 4A shows quadrant analyses by diagnosis. Pie charts showing the quadrants (I, II, III, IV) where participants from each disease group are located. FIG. 4B shows quadrant analyses by diagnosis. Pie charts showing the quadrants (I, II, III, IV) where participants from each disease group are located. FIG. 4C shows quadrant analyses by diagnosis. Pie charts showing the quadrants (I, II, III, IV) where participants from each disease group are located. FIG. 4D shows quadrant analyses by diagnosis. Pie charts showing the quadrants (I, II, III, IV) where participants from each disease group are located. FIG. 4E shows quadrant analyses by diagnosis. Pie charts showing the quadrants (I, II, III, IV) where participants from each disease group are located. FIG. 4F shows quadrant analyses by diagnosis. Pie charts showing the quadrants (I, II, III, IV) where participants from each disease group are located.

FIG. 5A, FIG. 5B, and FIG. 5C show amyloid and tau PET Imaging by quadrant. FIG. 5A shows amyloid PET imaging measured by PiB SUVR is significantly and gradually increased in quadrant II > III > IV (ANOVA, p<0.0001). FIG. 5B shows amyloid PET imaging measured by AV45 SUVR is significantly and gradually increased in quadrant II > III > IV (ANOVA, p<0.0001). FIG. 5C shows tau PET imaging measured by AV1451 SUVR is only increased in quadrant II (ANOVA, p<0.0001). There was no participant with imaging data in quadrant I. ANOVA *p<0.05, **p<0.01, ***p<0.001, ****p<0.0001.

FIG. 6 graphically depicts CSF Aβ 42/40 and CSF pT217/T217 correlate in quadrant III and IV. CSF pT217/T217 negatively correlates with CSF Ab 42/40 in quadrant III and IV below cutoff for ptau positivity (Pearson correlation r=0.556, p<0.0001).

FIG. 7A, FIG. 7B, FIG. 7C and FIG. 7D graphically depict Subcategory of diagnosis are shown from FIG. 2. FIG. 7A shows CSF Aβ 42/40 is significantly decreased in AD and AD focal. FIG. 7B shows CSF pT217/T217 is significantly increased in AD and AD focal. FIG. 7C shows CSF pT217/T217 × CSF Aβ 42/40, is significantly increased in AD and MAPT R406W mutation carriers. FIG. 7D shows CSF pT217/T217 divided by CSF Aβ 42/40, is significantly increased in AD and AD focal. ANOVA *p<0.05, **p<0.01, ***p<0.001, ****p<0.0001.

FIG. 8A, FIG. 8B, FIG. 8C and FIG. 8D graphically depict quadrant Analyses using CSF Ab 42/40 and CSF total tau and ptau. CSF concentrations of pT217 (FIG. 8A), pT181 (FIG. 8B), total tau (FIG. 8C) do not separate MAPT R406W mutation carriers or other groups. FIG. 8A shows CSF pT181/T181 ratio does not separate MAPT R406W mutation carriers. However, sporadic bvFTD may have lower CSF pT181/T181 in quadrant IV.

FIG. 9A, FIG. 9B, FIG. 9C and FIG. 9D graphically depict IP/MS CSF total tau and ptau in subgroups of tauopathies. FIG. 9A and FIG. 9B show AD has significantly increased CSF pT217 and pT181 concentrations. FIG. 9C shows total CSF tau is significantly increased in AMC, AD, AD focal, CBS PSP continuum than YNC. FIG. 9D show sporadic bvFTD containing FTLD-tau, FTLD-TDP, FTLD-FUS has significantly lower CSF pT1811T181 than R406W (*p<0.05), AMC (***p<0.001), and AD/AD focal (****p<0.0001).

FIG. 10A and FIG. 10B show sporadic bvFTD containing FTLD-tau, FTLD-TDP, FTLD-FUS may be separated from Control, AD and other tauopathies with CSF pT181/T181. FIG. 10A show sporadic bvFTD (bvFTD) containing FTLD-tau, FTLD-TDP, FTLD-FUS has higher CSF Aβ 42/40 ratio and lower CSF pT1811T181 than Control, AD, and tauopathies containing CBS, PSP, and MAPT-FTD (P301L and R406W mutation carriers). *p<0.05, **p<0.01, ***p<0.001, ****p<0.0001. FIG. 10B show diagnostic values of CSF pT181/T181 in separating bvFTD from other cohort. Sporadic bvFTD can be separated from Control (AUC = 0.752) and PSP, CBS, and MAPT-FTD (AUC = 0.707).

FIG. 11 shows all large portion of the participants identified as CBD or PSP have higher pT217/T217 than observed in AD samples.

FIG. 12 shows pT181/T181 values in AD and non-AD tauopathies.

FIG. 13 shows pT205/T205 values in AD and non-AD tauopathies.

FIG. 14 shows pS208/S208 values in AD and non-AD tauopathies.

FIG. 15 shows pT111/T111 values in AD and non-AD tauopathies.

FIG. 16 shows pT153/T153 values in AD and non-AD tauopathies.

FIG. 17A shows sporadic FTD containing FTD-tau, FTD-ALS, FTD-FUS can be separated from Control, AD and other tauopathies with CSF pT181/T181. FIG. 17B Sporadic FTD (sFTD) has higher CSF Ab 42/40 ratio and lower CSF pT181/T181 from other cohort in quadrant analyses. FIG. 17C sFTD has significantly lower CSF pT1811T181 than R406W (*p<0.05), AMC (***p<0.001), and AD/AD focal (****p<0.0001). FIG. 17D sFTD has significantly lower CSF pT181/T181 from Control, AD and CBS, PSP and familial FTD-MAPT (fFTD). sFTD can be separated from control (AUC = 0.75) and PSP, CBS, and fFTD (AUC = 0.72)

FIG. 18A shows MAPT R406W carriers have increased CSF pT217/T217 without Amyloid pathology and sporadic FTD has decreased pT181/T181. FIG. 18B All samples (n=265) were subjected to quadrant analyses with CSF Ab 42/40 ratio on x-axis and CSF tau measures on y-axis. FIG. 18C CSF pT217/T217 in increased in MAPT R406W mutation carriers. FIG. 18D (CSF Abeta 42/40 cutoff = 0.086, CSF pT217/T217 cutoff = 4.755. CSF pT217 absolute concentration does not separate MAPT R406W mutation carriers. FIG. 18E CSF pT181/T181 is decreased in sporadic FTD. CSF pT181 absolute concentration does not separate sporadic FTD. total tau does not separate any group.

FIG. 19A shows MAPT R406W carriers have increased pT217/T217 without Amyloid pathology and FIG. 19B sporadic FTD has decreased pT181/T181. FIG. 19C pT217/T217 is better than pT217 concentration alone to separate MAPT R406W carriers. FIG. 19D pT181/T181 is better than pT181 concentration alone to separate sporadic FTD not containing MAPT R406W and P301 L carriers. FIG. 19E Age in different disease cohort - FIG. 19F CSF total tau is partially age dependent.

### DETAILED DESCRIPTION

Tau protein aggregation into neurofibrillary tangles in the central nervous system contributes to the etiology of certain neurodegenerative disorders, including Alzheimer’s disease (AD). Though the mechanism of tau destabilization is not fully understood yet, tau protein has been found to be hyperphosphorylated in tau aggregates. The present disclosure encompasses use of the methods to quantify tau phosphorylation at specific amino acid residues to diagnose tauopathies, guide treatment decisions, select subjects for clinical trials, and evaluate the clinical efficacy of certain therapeutic interventions. Other aspects and iterations of the invention are described more thoroughly below.

### I. Definitions

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

An antibody, as used herein, refers to a complete antibody as understood in the art, i.e., consisting of two heavy chains and two light chains, and also to any antibody-like molecule that has an antigen binding region, including, but not limited to, antibody fragments such as Fab′, Fab, F(ab′)2, single domain antibodies, Fv, and single chain Fv. The term antibody also refers to a polyclonal antibody, a monoclonal antibody, a chimeric antibody and a humanized antibody. The techniques for preparing and using various antibody-based constructs and fragments are well known in the art. Means for preparing and characterizing antibodies are also well known in the art (See, e.g. Antibodies: A Laboratory Manual, Cold Spring Harbor Laboratory, 1988; herein incorporated by reference in its entirety).

As used herein, the term “aptamer” refers to a polynucleotide, generally a RNA or DNA that has a useful biological activity in terms of biochemical activity, molecular recognition or binding attributes. Usually, an aptamer has a molecular activity such as binging to a target molecule at a specific epitope (region). It is generally accepted that an aptamer, which is specific in it binding to a polypeptide, may be synthesized and/or identified by in vitro evolution methods. Means for preparing and characterizing aptamers, including by in vitro evolution methods, are well known in the art. See, for instance U.S. 7,939,313, herein incorporated by reference in its entirety.

The term “Aβ” refers to peptides derived from a region in the carboxy terminus of a larger protein called amyloid precursor protein (APP). The gene encoding APP is located on chromosome 21. There are many forms of Aβ that may have toxic effects: Aβ peptides are typically 37-43 amino acid sequences long, though they can have truncations and modifications changing their overall size. They can be found in soluble and insoluble compartments, in monomeric, oligomeric and aggregated forms, intracellularly or extracellularly, and may be complexed with other proteins or molecules. The adverse or toxic effects of Aβ may be attributable to any or all of the above noted forms, as well as to others not described specifically. For example, two such Aβ isoforms include Aβ40 and Aβ42; with the Aβ42 isoform being particularly fibrillogenic or insoluble and associated with disease states. The term “Aβ” typically refers to a plurality of Aβ species without discrimination among individual Aβ species. Specific Aβ species are identified by the size of the peptide, e.g., Aβ42, Aβ40, Aβ38 etc.

As used herein, the term “Aβ42/ Aβ40 value” means the ratio of the amount of Aβ42 in a sample obtained from a subject compared to the amount of Aβ40 in the same sample.

“Aβ amyloidosis” is defined as clinically abnormal Aβ deposition in the brain. A subject that is determined to have Aβ amyloidosis is referred to herein as “amyloid positive,” while a subject that is determined to not have Aβ amyloidosis is referred to herein as “amyloid negative.” There are accepted indicators of Aβ amyloidosis in the art. At the time of this disclosure, Aβ amyloidosis is directly measured by amyloid imaging (e.g., PiB PET, fluorbetapir, or other imaging methods known in the art) or indirectly measured by decreased cerebrospinal fluid (CSF) Aβ42 or a decreased CSF Aβ42/40 ratio. [¹¹C]PIB-PET imaging with mean cortical binding potential (MCBP) score > 0.18 is an indicator of Aβ amyloidosis, as is cerebral spinal fluid (CSF) Aβ42 concentration of about 1 ng/ml measured by immunoprecipitation and mass spectrometry (IP/MS)). Alternatively, a cut-off ratio for CSF Aβ42/40 that maximizes the accuracy in predicting amyloid-positivity as determined by PIB-PET can be used. Values such as these, or others known in the art and/or used in the examples, may be used alone or in combination to clinically confirm Aβ amyloidosis. See, for example, Klunk W E et al. Ann Neurol 55(3) 2004, Fagan A M et al. Ann Neurol, 2006, 59(3), Patterson et. al, Annals of Neurology, 2015, 78(3): 439-453, or Johnson et al., J. Nuc. Med., 2013, 54(7): 1011-1013, each hereby incorporated by reference in its entirety. Subjects with Aβ amyloidosis may or may not be symptomatic, and symptomatic subjects may or may not satisfy the clinical criteria for a disease associated with Aβ amyloidosis. Non-limiting examples of symptoms associated with Aβ amyloidosis may include impaired cognitive function, altered behavior, abnormal language function, emotional dysregulation, seizures, dementia, and impaired nervous system structure or function. Diseases associated with Aβ amyloidosis include, but are not limited to, Alzheimer’s Disease (AD), cerebral amyloid angiopathy (CAA), Lewy body dementia, and inclusion body myositis. Subjects with Aβ amyloidosis are at an increased risk of developing a disease associated with Aβ amyloidosis.

A “clinical sign of Aβ amyloidosis” refers to a measure of Aβ deposition known in the art. Clinical signs of Aβ amyloidosis may include, but are not limited to, Aβ deposition identified by amyloid imaging (e.g. PiB PET, fluorbetapir, or other imaging methods known in the art) or by decreased cerebrospinal fluid (CSF) Aβ42 or Aβ42/40 ratio. See, for example, Klunk WE et al. Ann Neurol 55(3) 2004, and Fagan AM et al. Ann Neurol 59(3) 2006, each hereby incorporated by reference in its entirety. Clinical signs of Aβ amyloidosis may also include measurements of the metabolism of Aβ, in particular measurements of Aβ42 metabolism alone or in comparison to measurements of the metabolism of other Aβ variants (e.g. Aβ37, Aβ38, Aβ39, Aβ40, and/or total Aβ), as described in U.S. Pat. Serial Nos. 14/366,831, 14/523,148 and 14/747,453, each hereby incorporated by reference in its entirety. Additional methods are described in Albert et al. Alzheimer’s & Dementia 2007 Vol. 7, pp. 170-179; McKhann et al., Alzheimer’s & Dementia 2007 Vol. 7, pp. 263-269; and Sperling et al. Alzheimer’s & Dementia 2007 Vol. 7, pp. 280-292, each hereby incorporated by reference in its entirety. Importantly, a subject with clinical signs of Aβ amyloidosis may or may not have symptoms associated with Aβ deposition. Yet subjects with clinical signs of Aβ amyloidosis are at an increased risk of developing a disease associated with Aβ amyloidosis.

A “candidate for amyloid imaging” refers to a subject that has been identified by a clinician as an individual for whom amyloid imaging may be clinically warranted. As a non-limiting example, a candidate for amyloid imaging may be a subject with one or more clinical signs of Aβ amyloidosis, one or more Aβ plaque associated symptoms, one or more CAA associated symptoms, or combinations thereof. A clinician may recommend amyloid imaging for such a subject to direct his or her clinical care. As another non-limiting example, a candidate for amyloid imaging may be a potential participant in a clinical trial for a disease associated with Aβ amyloidosis (either a control subject or a test subject).

An “Aβ plaque associated symptom” or a “CAA associated symptom” refers to any symptom caused by or associated with the formation of amyloid plaques or CAA, respectively, being composed of regularly ordered fibrillar aggregates called amyloid fibrils. Exemplary Aβ plaque associated symptoms may include, but are not limited to, neuronal degeneration, impaired cognitive function, impaired memory, altered behavior, emotional dysregulation, seizures, impaired nervous system structure or function, and an increased risk of development or worsening of Alzheimer’s disease or CAA. Neuronal degeneration may include a change in structure of a neuron (including molecular changes such as intracellular accumulation of toxic proteins, protein aggregates, etc. and macro level changes such as change in shape or length of axons or dendrites, change in myelin sheath composition, loss of myelin sheath, etc.), a change in function of a neuron, a loss of function of a neuron, death of a neuron, or any combination thereof. Impaired cognitive function may include but is not limited to difficulties with memory, attention, concentration, language, abstract thought, creativity, executive function, planning, and organization. Altered behavior may include, but is not limited to, physical or verbal aggression, impulsivity, decreased inhibition, apathy, decreased initiation, changes in personality, abuse of alcohol, tobacco or drugs, and other addiction-related behaviors. Emotional dysregulation may include, but is not limited to, depression, anxiety, mania, irritability, and emotional incontinence. Seizures may include but are not limited to generalized tonic-clonic seizures, complex partial seizures, and non-epileptic, psychogenic seizures. Impaired nervous system structure or function may include, but is not limited to, hydrocephalus, Parkinsonism, sleep disorders, psychosis, impairment of balance and coordination. This may include motor impairments such as monoparesis, hemiparesis, tetraparesis, ataxia, ballismus and tremor. This also may include sensory loss or dysfunction including olfactory, tactile, gustatory, visual and auditory sensation. Furthermore, this may include autonomic nervous system impairments such as bowel and bladder dysfunction, sexual dysfunction, blood pressure and temperature dysregulation. Finally, this may include hormonal impairments attributable to dysfunction of the hypothalamus and pituitary gland such as deficiencies and dysregulation of growth hormone, thyroid stimulating hormone, lutenizing hormone, follicle stimulating hormone, gonadotropin releasing hormone, prolactin, and numerous other hormones and modulators.

As used herein, the term “subject” refers to a mammal, preferably a human. The mammals include, but are not limited to, humans, primates, livestock, rodents, and pets. A subject may be waiting for medical care or treatment, may be under medical care or treatment, or may have received medical care or treatment.

As used herein, the term “control population,” “normal population” or a sample from a “healthy” subject refers to a subject, or group of subjects, who are clinically determined to not have a tauopathy or Aβ amyloidosis, or a clinical disease associated with Aβ amyloidosis (including but not limited to Alzheimer’s disease), based on qualitative or quantitative test results.

As used herein, the term “blood sample” refers to a biological sample derived from blood, preferably peripheral (or circulating) blood. The blood sample can be whole blood, plasma or serum, although plasma is typically preferred.

The term “isoform”, as used herein, refers to any of several different forms of the same protein variants, arising due to alternative splicing of mRNA encoding the protein, post-translational modification of the protein, proteolytic processing of the protein, genetic variations and somatic recombination. The terms “isoform” and “variant” are used interchangeably.

The term “tau” refers to a plurality of isoforms encoded by the gene MAPT (or homolog thereof), as well as species thereof that are C-terminally truncated in vivo, N-terminally truncated in vivo, post-translationally modified in vivo, or any combination thereof. As used herein, the terms “tau” and “tau protein” and “tau species” may be used interchangeably. In many animals, including but not limited to humans, non-human primates, rodents, fish, cattle, frogs, goats, and chicken, tau is encoded by the gene MAPT. In animals where the gene is not identified as MAPT, a homolog may be identified by methods well known in the art.

In humans, there are six isoforms of tau that are generated by alternative splicing of exons 2, 3, and 10 of MAPT. These isoforms range in length from 352 to 441 amino acids. Exons 2 and 3 encode 29-amino acid inserts each in the N-terminus (called N), and full-length human tau isoforms may have both inserts (2N), one insert (1N), or no inserts (ON). All full-length human tau isoforms also have three repeats of the microtubule binding domain (called R). Inclusion of exon 10 at the C-terminus leads to inclusion of a fourth microtubule binding domain encoded by exon 10. Hence, full-length human tau isoforms may be comprised of four repeats of the microtubule binding domain (exon 10 included: R1, R2, R3, and R4) or three repeats of the microtubule binding domain (exon 10 excluded: R1, R3, and R4). Human tau may or may not be post-translationally modified. For example, it is known in the art that tau may be phosphorylated, ubiquinated, glycosylated, and glycated. Human tau also may or may not be proteolytically processed in vivo at the C-terminus, at the N-terminus, or at the C-terminus and the N-terminus. Accordingly, the term “human tau” encompasses the 2N3R, 2N4R, 1N3R, 1N4R, 0N3R, and 0N4R isoforms, as well as species thereof that are C-terminally truncated in vivo, N-terminally truncated in vivo, post-translationally modified in vivo, or any combination thereof. Alternative splicing of the gene encoding tau similarly occurs in other animals.

The term “MTBR tau,” as used herein, refers to a tau protein, or a plurality of tau proteins, that comprise(s) two or more amino acids of the microtubule binding region (MTBR) of tau (e.g., amino acids 244-368 of tau-441, etc.).

A disease associated with tau deposition in the brain is referred to herein as a “tauopathy”. The term “tau deposition” is inclusive of all forms pathological tau deposits including but not limited to neurofibrillary tangles, neuropil threads, and tau aggregates in dystrophic neurites. Tauopathies known in the art include, but are not limited to, progressive supranuclear palsy (PSP), dementia pugilistica, chronic traumatic encephalopathy, frontotemporal dementia and parkinsonism linked to chromosome 17, Lytico-Bodig disease, Parkinson-dementia complex of Guam, tangle- predominant dementia, ganglioglioma and gangliocytoma, meningioangiomatosis, subacute sclerosing panencephalitis, lead encephalopathy, tuberous sclerosis, Hallervorden-Spatz disease, lipofuscinosis, Pick’s disease, corticobasal degeneration (CBD), argyrophilic grain disease (AGD), Frontotemporal lobar degeneration (FTLD), Alzheimer’s disease (AD), and frontotemporal dementia (FTD).

Tauopathies are classified by the predominance of tau isoforms found in the pathological tau deposits. Those tauopathies with tau deposits predominantly composed of tau with three MTBRs are referred to as “3R-tauopathies”. Pick’s disease is a non-limiting example of a 3R-tauopathy. For clarification, pathological tau deposits of some 3R-tauopathies may be a mix of 3R and 4R tau isoforms with 3R isoforms predominant. Intracellular neurofibrillary tangles (i.e. tau deposits) in brains of subjects with Alzheimer’s disease are generally thought to contain both approximately equal amounts of 3R and 4R isoforms. Those tauopathies with tau deposits predominantly composed of tau with four MTBRs are referred to as “4R-tauopathies”. PSP, CBD, and AGD are non-limiting examples of 4R-tauopathies, as are some forms of FTLD. Notably, pathological tau deposits in brains of some subjects with genetically confirmed FTLD cases, such as some V334M and R406W mutation carriers, show a mix of 3R and 4R isoforms.

A clinical sign of a tauopathy may be aggregates of tau in the brain, including but not limited to neurofibrillary tangles. Methods for detecting and quantifying tau aggregates in the brain are known in the art (e.g., tau PET using tau-specific ligands such as [¹⁸F]THK5317, [¹⁸F]THK5351, [¹⁸F]AV1451, [¹¹C]PBB3, [¹⁸F]MK-6240, [¹⁸F]RO-948, [¹⁸F]PI-2620, [¹⁸F]GTP1, [¹⁸F]PM-PBB3, and [¹⁸F]JNJ64349311, [¹⁸F]JNJ-067), etc.).

The terms “treat,” “treating,” or “treatment” as used herein, refers to the provision of medical care by a trained and licensed professional to a subject in need thereof. The medical care may be a diagnostic test, a therapeutic treatment, and/or a prophylactic or preventative measure. The object of therapeutic and prophylactic treatments is to prevent or slow down (lessen) an undesired physiological change or disease/disorder. Beneficial or desired clinical results of therapeutic or prophylactic treatments include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented. Accordingly, a subject in need of treatment may or may not have any symptoms or clinical signs of disease.

The phrase “tau therapy” collectively refers to any imaging agent, therapeutic treatment, and/or a prophylactic or preventative measure contemplated for, or used with, subjects at risk of developing a tauopathy, or subjects clinically diagnosed as having a tauopathy. Non-limiting examples of imaging agents include functional imaging agents (e.g. fluorodeoxyglucose, etc.) and molecular imaging agents (e.g., Pittsburgh compound B, florbetaben, florbetapir, flutemetamol, radiolabeled tau-specific ligands, radionuclide-labeled antibodies, etc.). Non-limiting examples of therapeutic agents include cholinesterase inhibitors, N-methyl D-aspartate (NMDA) antagonists, antidepressants (e.g., selective serotonin reuptake inhibitors, atypical antidepressants, aminoketones, selective serotonin and norepinephrine reuptake inhibitors, tricyclic antidepressants, etc.), gamma-secretase inhibitors, beta-secretase inhibitors, anti-Aβ antibodies (including antigen-binding fragments, variants, or derivatives thereof), anti-tau antibodies (including antigen- binding fragments, variants, or derivatives thereof), stem cells, dietary supplements (e.g. lithium water, omega-3 fatty acids with lipoic acid, long chain triglycerides, genistein, resveratrol, curcumin, and grape seed extract, etc.), antagonists of the serotonin receptor 6, p38alpha MAPK inhibitors, recombinant granulocyte macrophage colony-stimulating factor, passive immunotherapies, active vaccines (e.g. CAD106, AF20513, etc.), tau protein aggregation inhibitors (e.g. TRx0237, methylthionimium chloride, etc.), therapies to improve blood sugar control (e.g., insulin, exenatide, liraglutide pioglitazone, etc.), anti-inflammatory agents, phosphodiesterase 9A inhibitors, sigma-1 receptor agonists, kinase inhibitors, phosphatase activators, phosphatase inhibitors, angiotensin receptor blockers, CB1 and/or CB2 endocannabinoid receptor partial agonists, β-2 adrenergic receptor agonists, nicotinic acetylcholine receptor agonists, 5-HT2A inverse agonists, alpha-2c adrenergic receptor antagonists, 5-HT 1A and 1D receptor agonists, Glutaminyl-peptide cyclotransferase inhibitors, selective inhibitors of APP production, monoamine oxidase B inhibitors, glutamate receptor antagonists, AMPA receptor agonists, nerve growth factor stimulants, HMG-CoA reductase inhibitors, neurotrophic agents, muscarinic M1 receptor agonists, GABA receptor modulators, PPAR-gamma agonists, microtubule protein modulators, calcium channel blockers, antihypertensive agents, statins, and any combination thereof.

The phrase “Aβ therapies” collectively refers to any imaging agent or therapeutic agent contemplated for, or used with, subjects at risk of developing Aβ amyloidosis or AD, subjects diagnosed as having Aβ amyloidosis, or subjects diagnosed as having AD.

“Significantly deviate from the mean” refers to values that are at least 1 standard deviation, preferably at least 1.3 standard deviations, more preferably at least 1.5 standard deviations or even more preferably at least 2 standard deviations, above or below the mean.

### II. Measuring Tau Phosphorylation and Aβ in a Biological Sample

Methods of the present disclose comprise providing a biological sample obtained from a subject, isolating tau and/or Aβ, and measuring tau phosphorylation at one or more amino acid residue, Aβ40, Aβ42 and optionally one or more proteins.

Suitable biological samples include a blood sample or a cerebrospinal fluid (CSF) sample obtained from a subject. In some embodiments, the subject is a human. A human subject may be waiting for medical care or treatment, may be under medical care or treatment, or may have received medical care or treatment. In various embodiments, a human subject may be a healthy subject, a subject at risk of developing a neuro-degenerative disease, a subject with signs and/or symptoms of a neurodegenerative disease, or a subject diagnosed with a neurodegenerative disease. In further embodiments, the neurodegenerative disease may be a tauopathy. In specific examples, the tauopathy may be Alzheimer’s disease (AD), progressive supranuclear palsy (PSP), corticobasal degeneration (CBD), frontotemporal lobar degeneration (FTLD). In other embodiments, the subject is a laboratory animal. In a further embodiment, the subject is a laboratory animal genetically engineered to express human tau and optionally one or more additional human protein (e.g., human Aβ, human ApoE, etc.).

CSF may have been obtained by lumbar puncture with or without an indwelling CSF catheter. Multiple blood or CSF samples contemporaneously collected from the subject may be pooled. Blood may have been collected by venipuncture with or without an intravenous catheter, or by a finger stick (or the equivalent thereof). Once collected, blood or CSF samples may have been processed according to methods known in the art (e.g., centrifugation to remove whole cells and cellular debris; use of additives designed to stabilize and preserve the specimen prior to analytical testing; etc.). Blood or CSF samples may be used immediately or may be frozen and stored indefinitely. Prior to use in the methods disclosed herein, the biological sample may also have been modified, if needed or desired, to include protease inhibitors, isotope labeled internal standards, detergent(s) and chaotropic agent(s), and/or to deplete other analytes (e.g. proteins peptides, metabolites).

The size of the sample used can and will vary depending upon the sample type, the health status of the subject from whom the sample was obtained, and the analytes to be analyzed (in addition to tau). CSF samples volumes may be about 0.01 mL to about 5 mL, or about 0.05 mL to about 5 mL. In a specific example, the size of the sample may be about 0.05 mL to about 1 mL CSF. Plasma sample volumes may be about 0.01 mL to about 20 mL.

Isotope-labeling may be used as an internal standard to account for variability throughout sample processing and optionally to calculate an absolute concentration. Generally, an isotope-labeled, internal standard is added before significant sample processing, and it can be added more than once if needed.

Multiple isotope-labeled internal standards are described herein. All have a heavy isotope label incorporated into at least one amino acid residue. One or more full-length tau and/or Aβ isoforms may be used. Alternatively, or in addition, isoforms with post-translational modifications and/or peptide fragments may also be used, as is known in the art. Generally speaking, the labeled amino acid residues that are incorporated should increase the mass of the peptide without affecting its chemical properties, and the mass shift resulting from the presence of the isotope labels must be sufficient to allow the mass spectrometry method to distinguish the internal standard (IS) from endogenous analyte signals. As shown herein, suitable heavy isotope labels include, but are not limited to ²H, ¹³C, and ¹⁵N. Typically, about 1-10 ng of internal standard is usually sufficient.

An isolated tau sample, as used herein, refers to a composition comprising tau, wherein tau has been purified from blood or cerebrospinal fluid (CSF) obtained from a subject. In isolated tau samples of the present disclosure, tau has been either partially or completely purified from blood or CSF. Methods for purifying tau from blood or CSF are known in the art and include, but are not limited to, selective precipitation, size-exclusion chromatography, ion-exchange chromatography, and affinity purification. Suitable methods concentrate both phosphorylated tau and unphosphorylated tau from blood or CSF.

Thus, the methods of the present disclosure may comprise a step wherein one or more protein is depleted from a sample. The term “deplete” means to diminish in quantity or number. Accordingly, a sample depleted of a protein may have any amount of the protein that is measurably less than the amount in the original sample, including no amount of the protein.

Protein(s) may be depleted from a sample by a method that specifically targets one or more protein, for example by affinity depletion, solid phase extraction, or other method known in the art. Targeted depletion of a protein, or multiple proteins, may be used in situations where downstream analysis of that protein is desired (e.g., identification, quantification, analysis of post-translation modifications, etc.). For instance, Aβ peptides may be identified and quantified by methods known in the art following affinity depletion of Aβ with a suitable epitope-binding agent. As another non-limiting example, apolipoprotein E (ApoE) status may be determined by methods known in the art following affinity depletion of ApoE and identification of the ApoE isoform. Targeted depletion may also be used to isolate other proteins for subsequent analysis including, but not limited to, apolipoprotein J, synuclein, soluble amyloid precursor protein, alpha-2 macro-globulin, S100B, myelin basic protein, an interleukin, TNF, TREM-2, TDP-43, YKL-40, VILIP-1, NFL, prion protein, pNFH, and DJ-1.

In some embodiments, targeted depletion may occur by affinity depletion. Affinity depletion refers to methods that deplete a protein of interest from a sample by virtue of its specific binding properties to a molecule. Typically, the molecule is a ligand attached to a solid support, such as a bead, resin, tissue culture plate, etc. (referred to as an immobilized ligand). Immobilization of a ligand to a solid support may also occur after the ligand-protein interaction occurs. Suitable ligands include antibodies, aptamers, and other epitope-binding agents. The molecule may also be a polymer or other material that selectively absorbs a protein of interest. As a non-limiting example, polyhydroxymethylene substituted by fat oxethylized alcohol (e.g., PHM-L LIPOSORB, Sigma Aldrich) may be used to selectively absorb lipoproteins (including ApoE) from serum. Two or more affinity depletion agents may be combined to sequentially or simultaneously deplete multiple proteins.

Alternatively, protein(s) may be depleted from a sample by a more general method, for example by ultrafiltration or protein precipitation with an acid, an organic solvent or a salt. Generally speaking, these methods are used to reliably reduce high abundance and high molecular weight proteins, which in turn enriches for low molecular weight and/or low abundance proteins and peptides (e.g., tau, Aβ, etc.).

In some embodiments, proteins may be depleted from a sample by precipitation. Briefly, precipitation comprises adding a precipitating agent to a sample and thoroughly mixing, incubating the sample with precipitating agent to precipitate proteins, and separating the precipitated proteins by centrifugation or filtration. The resulting supernatant may then be used in downstream applications. The amount of the reagent needed may be experimentally determined by methods known in the art. Suitable precipitating agents include perchloric acid, trichloroacetic acid, acetonitrile, methanol, and the like. In an exemplary embodiment, proteins are depleted from a sample by acid precipitation. In a further embodiment, proteins are depleted from a sample by acid precipitation using perchloric acid.

Two or more methods from one or both of the above approaches may be combined to sequentially or simultaneously deplete multiple proteins. For instance, one or more proteins may be selectively depleted (targeted depletion) followed by depletion of high abundance / molecular weight proteins. Alternatively, high abundance / molecular weight proteins may be first depleted followed by targeted depletion of one or more proteins. In still another alternative, high abundance /molecular weight proteins may be first depleted followed by a first round of targeted depletion of one or more proteins and then a second round of targeted depletion of one or more different protein(s) than targeted in the first round. Other iterations will be readily apparent to a skilled artisan.

In an exemplary embodiment, isolated tau samples of the present disclosure comprise tau that has been purified from blood or CSF by affinity purification. Affinity purification refers to methods that purify a protein of interest by virtue of its specific binding properties to an immobilized ligand. Typically, an immobilized ligand is a ligand attached to a solid support, such as a bead, resin, tissue culture plate, etc. Suitable ligands specifically bind both phosphorylated and unphosphoryated tau. In one example, a suitable ligand may bind an epitope within the mid domain of tau. In another example, a suitable ligand may bind an epitope within the N-terminus of tau, preferably within amino acids 1 to 35 of tau. In another example, a suitable ligand may bind an epitope within the MTBR of tau. In another example, a suitable ligand may bind an epitope within the C-terminus of tau. In still further embodiments, tau may be affinity purified from blood or CSF using two or more immobilized ligands. In one example, an immobilized ligand binds an epitope within the N-terminus of tau and another immobilized ligand binds an epitope within the mid domain of tau. In another example, an immobilized ligand binds an epitope within the MTBR of tau and another immobilized ligand binds an epitope within the mid domain of tau. In another example, an immobilized ligand binds an epitope within the C-terminus of tau and another immobilized ligand binds an epitope within the mid domain of tau. In another example, an immobilized ligand binds an epitope within the C-terminus of tau and another immobilized ligand binds an epitope within the N-terminus of tau. In another example, an immobilized ligand binds an epitope within the MTBR of tau and another immobilized ligand binds an epitope within the N-terminus of tau. In another example, an immobilized ligand binds an epitope within the MTBR of tau and another immobilized ligand binds an epitope within the C-terminus of tau. In each of the above embodiments, the ligand may be an antibody or an aptamer.

In each of the above embodiments, the epitope binding agent may comprise an antibody or an aptamer. In some embodiments, the epitope-binding agent that specifically binds to amyloid beta is HJ5.1, or is an epitope-binding agent that binds the same epitope as HJ5.1 and/or competitively inhibits HJ5.1. In some embodiments, the epitope-binding agent that specifically binds to that specifically binds to an epitope within amino acids 1 to 103 of tau-441, inclusive, is HJ8.5, or is an epitope-binding agent that binds the same epitope as HJ8.5 and/or competitively inhibits HJ8.5. In some embodiments, the epitope-binding agent that specifically binds to that specifically binds to an epitope within amino acids 104 to 221 of tau-441, inclusive, is Tau1, or is an epitope-binding agent that binds the same epitope as Tau1 and/or competitively inhibits Tau1. Methods for identifying epitopes to which an antibody specifically binds, and assays to evaluate competitive inhibition between two antibodies, are known in the art.

Phosphorylation of specific amino acids (i.e. “sites”) in tau results in phosphorylated tau (p-tau) isoforms. Methods of the present disclosure provide means to measure the stoichiometry of phosphorylation at one or more specific amino acids of tau. In some embodiments, methods herein comprise measuring tau phosphorylation at one or more residue chosen from T111, S113, T181, S199, S202, S208, T153, T175, T205, S214, T217, and T231. In some embodiments, methods herein comprise measuring tau phosphorylation at one or more residue chosen from T111, T181, T153, and T217. In other embodiments, methods herein comprise measuring tau phosphorylation at one or more residue chosen from T181, and T217. In other embodiments, methods herein comprise measuring tau phosphorylation at one or more residue that includes T217. In other embodiments, methods herein comprise measuring tau phosphorylation at one or more residue that includes T181. In other embodiments, methods herein comprise measuring tau phosphorylation at two or more residues that include T181 and T217. In other embodiments, methods herein comprise measuring tau phosphorylation at three or more residues that include T181 and T217.

Applicants developed a highly sensitive and specific mass spectrometry (MS) method using parallel reaction monitoring (PRM) to discover tau phosphorylation sites and initially quantify the abundance of phosphorylation sites in isolated tau proteins. However, the present disclosure is not limited to any one particular method to quantitatively assess site-specific phosphorylation of tau. Suitable methods should discriminate tau isoforms that differ only in the phosphorylation status of a single amino acid, discriminate p-tau isoforms that are phosphorylated at different amino acids, and quantify changes in phosphorylation occurring at specific sites independently from the global change in total tau. Three approaches to quantify changes in phosphorylation stoichiometry occurring at specific sites independently from the global change in total tau are detailed in the examples: 1) relative comparison between phosphorylated peptide isomers, which can be used to estimate the relative abundance of each phosphorylated peptide sharing the same sequence; 2) normalizing phosphorylated peptides with any peptide from the tau protein as reference; and 3) absolute quantitation using internal synthetic labeled standards for each phosphorylated and non-phosphorylated peptide, where absolute quantitation values for each phosphorylated peptide is normalized with any absolute quantitation value obtained for any peptide from the tau protein. All three approaches use internal normalization for comparing relative phosphorylation changes for each site. Other methods known in the art may also be used.

In an exemplary embodiment, site-specific phosphorylation of tau is measured by high-resolution mass spectrometry. Suitable types of mass spectrometers are known in the art. These include, but are not limited to, quadrupole, time-of-flight, ion trap and Orbitrap, as well as hybrid mass spectrometers that combine different types of mass analyzers into one architecture (e.g., Orbitrap Fusion™ Tribrid™ Mass Spectrometer from ThermoFisher Scientific). Additional processing of an isolated tau sample may occur prior to MS analysis. For example, tau may be proteolytically digested. Suitable proteases include, but are not limited to, trypsin, Lys-N, Lys-C, and Arg-N. When affinity purification is used to produce an isolated tau sample, digestion may occur after eluting tau from the immobilized ligand or while tau is bound. Following one or more clean-up steps, digested tau peptides may be separated by a liquid chromatography system inter-faced with a high-resolution mass spectrometer. The chromatography system may be optimized by routine experimentation to produce a desired LC-MS pattern. A wide array of LC-MS techniques may be used to quantitatively analysis site-specific tau phosphorylation. Non-limiting examples include selected-reaction monitoring, parallel-reaction monitoring, selected-ion monitoring, and data-independent acquisition. As stated above, all quantitative assessments of site-specific tau phosphorylation should account for global changes in total tau. In an exemplary embodiment, a mass spectrometry protocol outlined in the Examples is used.

“Total tau,” as used herein refers to all tau isoforms in a given sample. Tau can be found in soluble and insoluble compartments, in monomeric and aggregated forms, in ordered or disordered structures, intracellularly and extracellularly, and may be complexed with other proteins or molecules. Accordingly, the source of the biological sample (e.g., brain tissue, CSF, blood, etc.) and any downstream processing of the biological sample will affect the totality of tau isoforms in a given sample.

Total tau may be measured by monitoring abundance of unmodified tau peptides. For each phosphorylated tau site, a tau peptide sharing the common amino acid sequence with the phosphorylated peptide of interest may preferentially be used to measure total tau level, but any peptide from the tau sequence can be used. Tau peptides measurement can be performed by mass spectrometry and accuracy of the measurement can be improved by using labeled internal standards as reference. Alternatively, total tau can be measured by immunoassays or other method quantifying tau concentration.

### III. Uses of Ptau and Aβ Measurements

The present disclosure also encompasses the use of measurements of ptau and Aβ species (e.g., pT217/T217, pT181/T181, and Aβ 42/40) in blood or CSF as biomarkers of pathological features and/or clinical symptoms of tauopathies in order to diagnose, stage, choose treatments appropriate for a given disease stage, and modify a given treatment regimen (e.g., change a dose, switch to a different drug or treatment modality, etc.). The pathological feature may be an aspect of tau pathology (e.g., amount of tau deposition, presence / absence of a post-translational modification, amount of a post-translation modification, etc.). Alternatively, or in addition to tau deposition, a pathological feature may be tau-independent. For instance, amyloid beta (Aβ) deposition in the brain or in arteries of the brain when the tauopathy is Alzheimer’s disease. The clinical symptom may be dementia, as measured by a clinically validated instrument (e.g., MMSE, CDR-SB, etc.), or any other clinical symptom associated with the tauopathy. Also contemplated is the use of measurements of ptau and Aβ species in blood or CSF as biomarkers of other pathological features and clinical symptoms known in the art for 3R- and 4R- tauopathies. Advantageously, ptau and Aβ species, including but not limited to pT217/T217, pT181/T181, and Aβ 42/40, not only discriminate a disease state from a healthy state, but also discriminate between the various tauopathies.

Accordingly, in one aspect, the present disclosure provides a method for measuring tauopathy-related pathology in a subject, the method comprising quantifying one or more ptau and Aβ species in a biological sample obtained from a subject, such as a blood sample or a CSF sample, wherein the amount(s) of the quantified ptau and Aβ species is/are a representation of tauopathy-related pathology in the brain of the subject. The tauopathy may be a 3R-tauopathy, a mixed 3R/4R-tauopathy, or a 4R-tauopathy. The disease-related pathology may be tau deposition, tau post-translational modification, amyloid plaques in the brain and/or arteries of the brain, or other pathological feature known in the art. The subject may or may not have clinical symptoms of the tauopathy. In preferred embodiments, at least one ptau species quantified is pT217. In further embodiments, two or more ptau species quantified are pT217 and pT181. In still further embodiments, the Aβ species quantified are Aβ 40 and Aβ 42 species. In still yet further embodiments, pT217/T217 and Aβ 42/40 and optionally pT1811T181 are quantified. In yet further embodiments, a composite pT217/T217 × Aβ 42/40 value is quantified.

In another aspect, the present disclosure provides a method for diagnosing a subject having a symptom of a tauopathy, the method comprising quantifying one or more ptau and Aβ species in a biological sample obtained from a subject, such as a blood sample or a CSF sample, and diagnosing a tauopathy when the quantified ptau and Aβ species deviate from a control population that does not have clinical signs or symptoms of a tauopathy and is amyloid negative as measured by PET imaging and/or Aβ42/40 measurement in CSF or deviate or are similar to a the same quantified ptau and Ab species from a population diagnosed with a 3R-tauopathy, a mixed 3R/4R-tauopathy, or a 4R-tauopathy. In preferred embodiments, at least one ptau species quantified is pT217. In further embodiments, two or more ptau species quantified are pT217 and pT181. In still further embodiments, the Aβ species quantified are Aβ 40 and Aβ 42 species. In still yet further embodiments, pT217/T217 and Aβ 42/40 and optionally pT1811T181 are quantified. In yet further embodiments, a composite pT217/T217 × Aβ 42/40 value is quantified.

In another aspect, the present disclosure provides a method for measuring tauopathy disease stability in a subject, the method comprising quantifying one or more ptau and Aβ species in a first biological sample obtained from a subject and then in a second biological sample obtained from the same subject at a later time (e.g., weeks, months or years later), and calculating the difference between the quantified ptau and Aβ species between the samples, wherein a statistically significant increase in the quantified ptau species in the second sample indicates disease progression, a statistically significant decrease in the quantified ptau species in the second sample indicates disease improvement, and no change indicates stable disease. The tauopathy may be a 3R-tauopathy, a mixed 3R/4R-tauopathy, or a 4R-tauopathy. The subject may or may not have clinical symptoms of disease, and may or may not be receiving a tau therapy. In some examples, a tau therapy is administered one or more times to the subject in the period of time between collection of the first and second biological sample, and the measure of disease stability is an indication of the effectiveness, or lack thereof, of the tau therapy. In preferred embodiments, at least one ptau species quantified is pT217. In further embodiments, two or more ptau species quantified are pT217 and pT181. In still further embodiments, the Aβ species quantified are Aβ 40 and Aβ 42 species. In still yet further embodiments, pT217/T217 and Aβ 42/40 and optionally pT1811T181 are quantified. In yet further embodiments, a composite pT217/T217 × Aβ 42/40 value is quantified.

In another aspect, the present disclosure provides a method for treating a subject with a tauopathy, the method comprising quantifying one or more ptau and Aβ species in a biological sample obtained from a subject, such as a blood sample or a CSF sample; and providing a tau therapy to the subject to improve a measurement of disease-related pathology or a clinical symptom, wherein the subject has a quantified ptau species at least 1 standard deviation, preferably at least 1.3 standard deviations, more preferably at least 1.5 standard deviations or even more preferably at least 2 standard deviations, above or below the mean (i.e., differs by 1σ, 1.3σ, 1.5σ, or 1.5σ, respectively, where σ is the standard deviation defined by the normal distribution measured in a control population does not have clinical signs or symptoms of a tauopathy and that is amyloid negative as measured by PET imaging and/or Aβ42/40 measurement in CSF. In addition to using a threshold (e.g. at least 1 standard deviation above or below the mean), in some embodiments the extent of change above or below the mean may be used as criteria for treating a subject. The tauopathy may be a 3R-tauopathy, a mixed 3R/4R-tauopathy, or a 4R-tauopathy. The measurement of disease-related pathology may be tau deposition as measured by PET imaging, tau post-translational modification as measured by mass spectrometry or other suitable method, amyloid plaques in the brain or arteries of the brain as measured by PET imaging, amyloid plaques as measured by Aβ42/40 in CSF, or other pathological features known in the art. The clinical symptom may be dementia, as measured by a clinically validated instrument (e.g., MMSE, CDR-SB, etc.) or other clinical symptoms known in the art for 3R- and 4R- tauopathies. In preferred embodiments, at least one ptau species quantified is pT217. In further embodiments, two or more ptau species quantified are pT217 and pT181. In still further embodiments, the Aβ species quantified are Aβ 40 and Aβ 42 species. In still yet further embodiments, pT217/T217 and Aβ 42/40 and optionally pT1811T181 are quantified. In yet further embodiments, a composite pT217/T217 × Aβ 42/40 value is quantified. Many tau therapies target a specific pathophysiological change. For instance, Aβ targeting therapies are generally designed to decrease Aβ production, antagonize Aβ aggregation or increase brain Aβ clearance; tau targeting therapies are generally designed to alter tau phosphorylation patterns, antagonize tau aggregation (general antagonism of tau or antagonism of a specific tau isoform), or increase NFT clearance; a variety of therapies are designed to reduce CNS inflammation or brain insulin resistance; etc. However, not all tauopathies share the same pathophysiological changes. Therefore, the efficacy of these various tau therapies can be improved by administering them to subjects that are correctly identified as having a 3R-tauopathy, a mixed 3R/4R-tauopathy, or a 4R-tauopathy.

Suitable biological samples and methods of measuring tau phosphorylation and Aβ species are described in Section II, the disclosures of which are incorporated into this section by reference.

In a specific embodiment, the present disclosure provides a method for discriminating a MAPT R406W tauopathy, the method comprising providing a processed CSF or blood sample obtained from a subject, wherein the CSF or blood sample is enriched for one or more ptau and Aβ species; and quantifying, in the processed sample, pT217/T217 value, and Aβ 42/40 value; wherein the pT217/T217 value, and Aβ 42/40 value discriminates a MAPT R406W tauopathy from Alzheimer’s disease and a healthy state.

In another specific embodiment, the present disclosure provides a method for discriminating a MAPT R406W tauopathy, the method comprising providing a processed CSF or blood sample obtained from a subject, wherein the CSF or blood sample is enriched for one or more ptau and Aβ species; and quantifying, in the processed sample, pT217/T217 value and Aβ 42/40 value; wherein an increase in the pT217/T217 value and a normal Aβ 42/40 value discriminates a MAPT R406W tauopathy from a healthy state. In one aspect, the calculated change(s) significantly deviate from the mean in a control population without brain amyloid plaques as measured by PET imaging. “Significantly deviate from the mean” includes values that are at least 1 standard deviation, preferably at least 1.3 standard deviations or more preferably at least 1.5 standard deviations or even more preferably at least 2 standard deviations, above or below the mean (i.e., 1σ, 1.3σ, 1.5σ, or 1.5σ, respectively, where σ is the standard deviation defined by the normal distribution measured in a control population without brain amyloid plaques as measured by PET imaging). In addition to using a threshold (e.g. at least 1 standard deviation above or below the mean), in some embodiment the extent of change above or below the mean may be used to diagnose a subject. A sample can be obtained from a subject that may or may not have a clinical diagnosis. In further embodiments, a subject may carry one of the gene mutations known to cause a tauopathy. In alternative embodiments, a subject may not carry a gene mutation known to cause a tauopathy.

In another specific embodiment, the present disclosure provides a method for discriminating a MAPT R406W tauopathy, the method comprising providing a processed CSF or blood sample obtained from a subject, wherein the CSF or blood sample is enriched for one or more ptau and Aβ species; and quantifying, in the processed sample, pT217/T217 value and Aβ 42/40 value; wherein a decrease in the pT217/T217 value and an increase Aβ 42/40 value discriminates a MAPT R406W tauopathy from AD. In one aspect, the calculated change(s) significantly deviate from the mean in a control population without brain amyloid plaques as measured by PET imaging. “Significantly deviate from the mean” includes values that are at least 1 standard deviation, preferably at least 1.3 standard deviations or more preferably at least 1.5 standard deviations or even more preferably at least 2 standard deviations, above or below the mean (i.e., 1σ, 1.3σ, 1.5σ, or 1.5σ′ respectively, where σ is the standard deviation defined by the normal distribution measured in an AD population with brain amyloid plaques as measured by PET imaging). In addition to using a threshold (e.g. at least 1 standard deviation above or below the mean), in some embodiment the extent of change above or below the mean may be used to diagnose a subject. A sample can be obtained from a subject that may or may not have a clinical diagnosis. In further embodiments, a subject may carry one of the gene mutations known to cause a tauopathy. In alternative embodiments, a subject may not carry a gene mutation known to cause a tauopathy.

In another specific embodiment, the present disclosure provides a method for discriminating a MAPT R406W tauopathy, the method comprising providing a processed CSF or blood sample obtained from a subject, wherein the CSF or blood sample is enriched for one or more ptau and Aβ species; and quantifying, in the processed sample, a composite pT217/T217 × Aβ 42/40 value; wherein the pT217/T217 × Aβ 42/40 value discriminates a MAPT R406W tauopathy from Alzheimer’s disease, 4R-tauopathy and a healthy state.

In another specific embodiment, the present disclosure provides a method for discriminating a MAPT R406W tauopathy, the method comprising providing a processed CSF or blood sample obtained from a subject, wherein the CSF or blood sample is enriched for one or more ptau and Aβ species; and quantifying, in the processed sample, a composite pT217/T217 × Aβ 42/40 value; wherein an increased pT217/T217 × Aβ 42/40 value discriminates a MAPT R406W tauopathy from a healthy state. In one aspect, the calculated change(s) significantly deviate from the mean in a control population without brain amyloid plaques as measured by PET imaging. “Significantly deviate from the mean” includes values that are at least 1 standard deviation, preferably at least 1.3 standard deviations or more preferably at least 1.5 standard deviations or even more preferably at least 2 standard deviations, above or below the mean (i.e., 1σ, 1.3σ, 1.5σ, or 1.5σ, respectively, where σ is the standard deviation defined by the normal distribution measured in an control population without brain amyloid plaques as measured by PET imaging). In addition to using a threshold (e.g. at least 1 standard deviation above or below the mean), in some embodiment the extent of change above or below the mean may be used to diagnose a subject. A sample can be obtained from a subject that may or may not have a clinical diagnosis. In further embodiments, a subject may carry one of the gene mutations known to cause a tauopathy.

In another specific embodiment, the present disclosure provides a method for discriminating a MAPT R406W tauopathy, the method comprising providing a processed CSF or blood sample obtained from a subject, wherein the CSF or blood sample is enriched for one or more ptau and Aβ species; and quantifying, in the processed sample, a composite pT217/T217 × Aβ 42/40 value; wherein an increased pT217/T217 × Aβ 42/40 value discriminates a MAPT R406W tauopathy from AD. In one aspect, the calculated change(s) significantly deviate from the mean in an AD population with brain amyloid plaques as measured by PET imaging. “Significantly deviate from the mean” includes values that are at least 1 standard deviation, preferably at least 1.3 standard deviations or more preferably at least 1.5 standard deviations or even more preferably at least 2 standard deviations, above or below the mean (i.e., 1σ, 1.3σ, 1.5σ, or 1.5σ, respectively, where σ is the standard deviation defined by the normal distribution measured in an AD population with brain amyloid plaques as measured by PET imaging). In addition to using a threshold (e.g. at least 1 standard deviation above or below the mean), in some embodiment the extent of change above or below the mean may be used to diagnose a subject. A sample can be obtained from a subject that may or may not have a clinical diagnosis. In further embodiments, a subject may carry one of the gene mutations known to cause a tauopathy.

In another specific embodiment, the present disclosure provides a method for discriminating a MAPT R406W tauopathy, the method comprising providing a processed CSF or blood sample obtained from a subject, wherein the CSF or blood sample is enriched for one or more ptau and Aβ species; and quantifying, in the processed sample, a composite pT217/T217 × Aβ 42/40 value; wherein an increased pT217/T217 × Aβ 42/40 value discriminates a MAPT R406W tauopathy from a 4R-tauopathy. In one aspect, the calculated change(s) significantly deviate from the mean in a 4R-tauopathy population without brain amyloid plaques as measured by PET imaging. “Significantly deviate from the mean” includes values that are at least 1 standard deviation, preferably at least 1.3 standard deviations or more preferably at least 1.5 standard deviations or even more preferably at least 2 standard deviations, above or below the mean (i.e., 1σ, 1.3σ, 1.5σ, or 1.5σ, respectively, where σ is the standard deviation defined by the normal distribution measured in a 4R-tauopathy population without brain amyloid plaques as measured by PET imaging). In addition to using a threshold (e.g. at least 1 standard deviation above or below the mean), in some embodiment the extent of change above or below the mean may be used to diagnose a subject. A sample can be obtained from a subject that may or may not have a clinical diagnosis. In further embodiments, a subject may carry one of the gene mutations known to cause a tauopathy.

In another specific embodiment, the present disclosure provides a method for discriminating a sporadic frontotemporal dementia (FTD), the method comprising providing a processed CSF or blood sample obtained from a subject, wherein the CSF or blood sample is enriched for one or more ptau species; and quantifying, in the processed sample, pT181/T181 value, wherein the pT1811T181 value discriminates a sporadic FTD from Alzheimer’s disease, other tauopathies and a healthy state.

In another specific embodiment, the present disclosure provides a method for discriminating a sporadic frontotemporal dementia (FTD), the method comprising providing a processed CSF or blood sample obtained from a subject, wherein the CSF or blood sample is enriched for one or more ptau species; and quantifying, in the processed sample, pT181/T181 value, wherein a decreased pT181/T181 value discriminates a sporadic FTD from Alzheimer’s disease. In one aspect, the calculated change(s) significantly deviate from the mean in a control population without brain amyloid plaques as measured by PET imaging. “Significantly deviate from the mean” includes values that are at least 1 standard deviation, preferably at least 1.3 standard deviations or more preferably at least 1.5 standard deviations or even more preferably at least 2 standard deviations, above or below the mean (i.e., 1σ, 1.3σ, 1.5σ, or 1.5σ, respectively, where σ is the standard deviation defined by the normal distribution measured in an AD population with brain amyloid plaques as measured by PET imaging). In addition to using a threshold (e.g. at least 1 standard deviation above or below the mean), in some embodiment the extent of change above or below the mean may be used to diagnose a subject. A sample can be obtained from a subject that may or may not have a clinical diagnosis. In further embodiments, a subject may carry one of the gene mutations known to cause a tauopathy. In alternative embodiments, a subject may not carry a gene mutation known to cause a tauopathy.

In another specific embodiment, the present disclosure provides a method for discriminating a sporadic frontotemporal dementia (FTD), the method comprising providing a processed CSF or blood sample obtained from a subject, wherein the CSF or blood sample is enriched for one or more ptau species; and quantifying, in the processed sample, pT181/T181 value, wherein a decreased pT181/T181 value discriminates a sporadic FTD from a healthy state. In one aspect, the calculated change(s) significantly deviate from the mean in a control population without brain amyloid plaques as measured by PET imaging. “Significantly deviate from the mean” includes values that are at least 1 standard deviation, preferably at least 1.3 standard deviations or more preferably at least 1.5 standard deviations or even more preferably at least 2 standard deviations, above or below the mean (i.e., 1σ, 1.3σ, 1.5σ, or 1.5σ, respectively, where σ is the standard deviation defined by the normal distribution measured in a control population without brain amyloid plaques as measured by PET imaging). In addition to using a threshold (e.g. at least 1 standard deviation above or below the mean), in some embodiment the extent of change above or below the mean may be used to diagnose a subject. A sample can be obtained from a subject that may or may not have a clinical diagnosis. In further embodiments, a subject may carry one of the gene mutations known to cause a tauopathy. In alternative embodiments, a subject may not carry a gene mutation known to cause a tauopathy.

In another specific embodiment, the present disclosure provides a method for discriminating a sporadic frontotemporal dementia (FTD), the method comprising providing a processed CSF or blood sample obtained from a subject, wherein the CSF or blood sample is enriched for one or more ptau species; and quantifying, in the processed sample, pT181/T181 value, wherein a decreased pT181/T181 value discriminates a sporadic FTD from a healthy state. In one aspect, the calculated change(s) significantly deviate from the mean in a non-sporadic FTD tauopathy population without brain amyloid plaques as measured by PET imaging. “Significantly deviate from the mean” includes values that are at least 1 standard deviation, preferably at least 1.3 standard deviations or more preferably at least 1.5 standard deviations or even more preferably at least 2 standard deviations, above or below the mean (i.e., 1σ, 1.3σ, 1.5σ, or 1.5σ, respectively, where σ is the standard deviation defined by the normal distribution measured in a non-sporadic FTD tauopathy population without brain amyloid plaques as measured by PET imaging). In addition to using a threshold (e.g. at least 1 standard deviation above or below the mean), in some embodiment the extent of change above or below the mean may be used to diagnose a subject. A sample can be obtained from a subject that may or may not have a clinical diagnosis. In further embodiments, a subject may carry one of the gene mutations known to cause a tauopathy. In alternative embodiments, a subject may or may not carry a gene mutation known to cause a tauopathy.

In another specific embodiment, the present disclosure provides a method for measuring MAPT R406W tauopathy disease progression in a subject, the method comprising providing a first processed CSF or blood sample obtained from a subject, wherein the first CSF or blood sample is enriched for one or more ptau and Aβ species; and quantifying, in the processed sample, pT217/T217 value, and Aβ 42/40 value; providing a second processed CSF or blood sample obtained from the subject after the first sample (e.g. days, weeks, months), wherein the second CSF or blood sample is enriched for the same ptau and Aβ species; and quantifying, in the processed sample, pT217/T217 value, and Aβ 42/40 value; and calculating the difference between the quantified pT217/T217 value and Aβ 42/40 value in the second sample and the first sample, wherein a statistically significant difference in the quantified pT217/T217 value, and Aβ 42/40 value in the second sample indicates progression of the subject’s disease.

In another specific embodiment, the present disclosure provides a method for measuring MAPT R406W tauopathy disease progression in a subject, the method comprising providing a first processed CSF or blood sample obtained from a subject, wherein the first CSF or blood sample is enriched for one or more ptau and Aβ species; and quantifying, in the processed sample, pT217/T217 value, and Aβ 42/40 value; providing a second processed CSF or blood sample obtained from the subject after the first sample (e.g. days, weeks, months), wherein the second CSF or blood sample is enriched for the same ptau and Aβ species; and quantifying, in the processed sample, pT217/T217 value, and Aβ 42/40 value; and calculating the difference between the quantified pT217/T217 value and Aβ 42/40 value in the second sample and the first sample, wherein no statistically significant difference in the quantified pT217/T217 value, and Aβ 42/40 value in the second sample indicates stability of the subject’s disease.

In another specific embodiment, the present disclosure provides a method for measuring MAPT R406W tauopathy disease progression in a subject, the method comprising providing a first processed CSF or blood sample obtained from a subject, wherein the first CSF or blood sample is enriched for one or more ptau and Aβ species; and quantifying, in the processed sample, a composite pT217/T217 × Aβ 42/40 value; providing a second processed CSF or blood sample obtained from the subject after the first sample (e.g. days, weeks, months), wherein the second CSF or blood sample is enriched for the same ptau and Aβ species; and quantifying, in the processed sample, a composite pT217/T217 × Aβ 42/40 value; and calculating the difference between the quantified composite pT217/T217 × Aβ 42/40 value in the second sample and the first sample, wherein a statistically significant difference in the quantified composite pT217/T217 × Aβ 42/40 value in the second sample indicates progression of the subject’s disease.

In another specific embodiment, the present disclosure provides a method for measuring MAPT R406W tauopathy disease progression in a subject, the method comprising providing a first processed CSF or blood sample obtained from a subject, wherein the first CSF or blood sample is enriched for one or more ptau and Aβ species; and quantifying, in the processed sample, a composite pT217/T217 × Aβ 42/40 value; providing a second processed CSF or blood sample obtained from the subject after the first sample (e.g. days, weeks, months), wherein the second CSF or blood sample is enriched for the same ptau and Aβ species; and quantifying, in the processed sample, a composite pT217/T217 × Aβ 42/40 value; and calculating the difference between the quantified composite pT217/T217 × Aβ 42/40 value in the second sample and the first sample, wherein no statistically significant difference in the quantified composite pT217/T217 × Aβ 42/40 value in the second sample indicates stability of the subject’s disease.

In another specific embodiment, the present disclosure provides a method for measuring sporadic frontotemporal dementia (FTD) disease progression in a subject, the method comprising providing a first processed CSF or blood sample obtained from a subject, wherein the first CSF or blood sample is enriched for one or more ptau species; and quantifying, in the processed sample, a pT181/T181 value; providing a second processed CSF or blood sample obtained from the subject after the first sample (e.g. days, weeks, months), wherein the second CSF or blood sample is enriched for the same ptau species; and quantifying, in the processed sample, a pT181/T181 value; and calculating the difference between the quantified pT1811T181 value in the second sample and the first sample, wherein a statistically significant difference in the quantified composite pT181/T181 value in the second sample indicates progression of the subject’s disease.

In another specific embodiment, the present disclosure provides a method for measuring sporadic frontotemporal dementia (FTD) disease progression in a subject, the method comprising providing a first processed CSF or blood sample obtained from a subject, wherein the first CSF or blood sample is enriched for one or more ptau species; and quantifying, in the processed sample, a composite pT181/T181 value; providing a second processed CSF or blood sample obtained from the subject after the first sample (e.g. days, weeks, months), wherein the second CSF or blood sample is enriched for the same ptau species; and quantifying, in the processed sample, a pT181/T181 value; and calculating the difference between the quantified pT181/T181 value in the second sample and the first sample, wherein no statistically significant difference in the quantified pT181/T181 value in the second sample indicates stability of the subject’s disease.

Alternatively or in addition to using a measurement of site-specific tau phosphorylation and Aβ species, optionally with a measurement of total tau, in any of the above embodiments, a ratio calculated from the measured phosphorylation level(s), or a ratio calculated from the measured phosphorylation level(s) and total tau, may be used. Both approaches are detailed in the examples. Mathematical operations other than a ratio may also be used. For instance, the examples use site-specific tau phosphorylation values in various statistical models (e.g., linear regressions, LME curves, LOESS curves, etc.) in conjunction with other known biomarkers (e.g. MAPT status, APOE ε4 status, age, sex, cognitive test scores, functional test scores, etc.). Selection of measurements and choice of mathematical operations may be optimized to maximize specificity of the method. For instance, diagnostic accuracy may be evaluated by area under the ROC curve and in some embodiments, an ROC AUC value of 0.7 or greater is set as a threshold (e.g., 0.7, 0.75, 0.8, 0.85, 0.9, 0.95, etc.).

Brain amyloid plaques in humans are routinely measured by amyloid-positron emission tomography (PET). For instance, ¹¹C-Pittsburgh compound B (PiB) PET imaging of cortical Aβ-plaques is commonly used to detect Aβ-plaque pathology. The standard uptake value ratio (SUVR) of cortical PiB-PET reliably identifies significant cortical Aβ-plaques and is used to classify subjects as PIB positive (SUVR ≥ 1.25) or negative (SUVR < 1.25). Accordingly, in the above embodiments, a control population without brain amyloid plaques as measured by PET imaging may refer to a population of subjects that have a cortical PiB-PET SUVR < 1.25. Other values of PiB binding (e.g., mean cortical binding potential) or analyses of regions of interest other than the cortical region may also be used to classify subjects as PIB positive or negative. Other PET imaging agents may also be used.

In another specific embodiment, the present disclosure provides a method for treating a subject in need thereof, the method comprising (a) providing a processed CSF or blood sample obtained from a subject, wherein the CSF or blood sample is enriched for one or more ptau and Aβ species; (b) quantifying, in the processed sample, pT217/T217 value and Aβ 42/40 value; and (c) administering a treatment to the subject to alter tau pathology, wherein the subject’s processed CSF or blood sample has quantified pT217/T217 value and Aβ 42/40 value, that differ by about 1.5σ or more, where σ is the standard deviation defined by the normal distribution measured in a control population that does not have clinical signs or symptoms of a tauopathy and is amyloid negative as measured by PET imaging, and wherein the amount of the quantified pT217/T217 value is a representation of tau pathology in a brain of a subject. In some embodiments, administering a treatment to the subject to alter tau pathology alters or stabilizes the amount of the quantified ptau species. In some embodiments the treatment is a pharmaceutical composition comprising a cholinesterase inhibitor, an N-methyl D-aspartate (NMDA) antagonist, an antidepressant (e.g., a selective serotonin reuptake inhibitor, an atypical antidepressant, an aminoketone, a selective serotonin and norepinephrine reuptake inhibitor, a tricyclic antidepressant, etc.), a gamma-secretase inhibitor, a beta-secretase inhibitor, an anti-Aβ antibody (including antigen-binding fragments, variants, or derivatives thereof), an anti-tau antibody (including antigen-binding fragments, variants, or derivatives thereof), an anti-TREM2 antibody (including antigen-binding fragments, variants or derivatives thereof, a TREM2 agonist, stem cells, dietary supplements (e.g. lithium water, omega-3 fatty acids with lipoic acid, long chain triglycerides, genistein, resveratrol, curcumin, and grape seed extract, etc.), an antagonist of the serotonin receptor 6, a p38alpha MAPK inhibitor, a recombinant granulocyte macrophage colony-stimulating factor, a passive immunotherapy, an active vaccine (e.g. CAD106, AF20513, etc.), a tau protein aggregation inhibitor (e.g. TRx0237, methylthionimium chloride, etc.), a therapy to improve blood sugar control (e.g., insulin, exenatide, liraglutide pioglitazone, etc.), an anti-inflammatory agent, a phosphodiesterase 9A inhibitor, a sigma-1 receptor agonist, a kinase inhibitor, a phosphatase activator, a phosphatase inhibitor, an angiotensin receptor blocker, a CB1 and/or CB2 endocannabinoid receptor partial agonist, a β-2 adrenergic receptor agonist, a nicotinic acetylcholine receptor agonist, a 5-HT2A inverse agonist, an alpha-2c adrenergic receptor antagonist, a 5-HT 1A and 1D receptor agonist, a Glutaminyl-peptide cyclotransferase inhibitor, a selective inhibitor of APP production, a monoamine oxidase B inhibitor, a glutamate receptor antagonist, a AMPA receptor agonist, a nerve growth factor stimulant, a HMG-CoA reductase inhibitor, a neurotrophic agent, a muscarinic M1 receptor agonist, a GABA receptor modulator, a PPAR-gamma agonist, a microtubule protein modulator, a calcium channel blocker, an antihypertensive agent, a statin, and any combination thereof. In an exemplary embodiment, a pharmaceutical composition may comprise a kinase inhibitor. Suitable kinase inhibitors may inhibit a thousand-and-one amino acid kinase (TAOK), CDK, GSK-3β, MARK, CDK5, or Fyn. In another exemplary embodiment, a pharmaceutical composition may comprise a phosphatase activator. As a non-limiting example, a phosphatase activator may increase the activity of protein phosphatase 2A. In some embodiments the treatment is a pharmaceutical composition comprising a tau targeting therapy, including but not limited to active pharmaceutical ingredients that alter tau phosphorylation patterns, antagonize tau aggregation, or increase clearance of pathological tau isoforms and/or aggregates. In some embodiments, the treatment is an anti-Aβ antibody, an anti-tau antibody, an anti-TREM2 antibody, a TREM2 agonist, a gamma-secretase inhibitor, a beta-secretase inhibitor, a kinase inhibitor, a phosphatase activator, a vaccine, or a tau protein aggregation inhibitor. In one embodiment, an increase in the pT217/T217 value and a normal Aβ 42/40 value relative to a healthy control indicates the subject be treated with a MAPT R406W therapy. In another embodiment, a decrease in the pT217/T217 value and an increase Aβ 42/40 value relative to an AD population indicates the subject be treated with a MAPT R406W therapy or tau therapy. In yet another embodiment, an increase in the pT217/T217 value and a decrease Aβ 42/40 value relative to an MAPT R406W population indicates the subject be treated with an AD therapy. In yet another embodiment, an increase in the composite pT217/T217 × Aβ 42/40 value relative to an control population or AD population indicates the subject be treated with a tau therapy.

In another specific embodiment, the present disclosure provides a method for treating a subject in need thereof, the method comprising (a) providing a processed CSF or blood sample obtained from a subject, wherein the CSF or blood sample is enriched for one or more ptau and Aβ species; (b) quantifying, in the processed sample, pT1811T181 value; and (c) administering a treatment to the subject to alter tau pathology, wherein the subject’s processed CSF or blood sample has quantified MTBR tau species, or ratios of the quantified MTBR tau species, that differ by about 1.5σ or more, where σ is the standard deviation defined by the normal distribution measured in a control population that does not have clinical signs or symptoms of a tauopathy and is amyloid negative as measured by PET imaging, and wherein the amount of the quantified ptau species is a representation of tau pathology in a brain of a subject. In some embodiments, administering a treatment to the subject to alter tau pathology alters or stabilizes the amount of the ptau species. In some embodiments the treatment is a pharmaceutical composition comprising a cholinesterase inhibitor, an N-methyl D-aspartate (NMDA) antagonist, an antidepressant (e.g., a selective serotonin reuptake inhibitor, an atypical antidepressant, an aminoketone, a selective serotonin and norepinephrine reuptake inhibitor, a tricyclic antidepressant, etc.), a gamma-secretase inhibitor, a beta-secretase inhibitor, an anti-Aβ antibody (including antigen-binding fragments, variants, or derivatives thereof), an anti-tau antibody (including antigen- binding fragments, variants, or derivatives thereof), an anti-TREM2 antibody (including antigen-binding fragments, variants or derivatives thereof, a TREM2 agonist, stem cells, dietary supplements (e.g. lithium water, omega-3 fatty acids with lipoic acid, long chain triglycerides, genistein, resveratrol, curcumin, and grape seed extract, etc.), an antagonist of the serotonin receptor 6, a p38alpha MAPK inhibitor, a recombinant granulocyte macrophage colony-stimulating factor, a passive immunotherapy, an active vaccine (e.g. CAD106, AF20513, etc.), a tau protein aggregation inhibitor (e.g. TRx0237, methylthionimium chloride, etc.), a therapy to improve blood sugar control (e.g., insulin, exenatide, liraglutide pioglitazone, etc.), an anti-inflammatory agent, a phosphodiesterase 9A inhibitor, a sigma-1 receptor agonist, a kinase inhibitor, a phosphatase activator, a phosphatase inhibitor, an angiotensin receptor blocker, a CB1 and/or CB2 endocannabinoid receptor partial agonist, a β-2 adrenergic receptor agonist, a nicotinic acetylcholine receptor agonist, a 5-HT2A inverse agonist, an alpha-2c adrenergic receptor antagonist, a 5-HT 1A and 1D receptor agonist, a Glutaminyl-peptide cyclotransferase inhibitor, a selective inhibitor of APP production, a monoamine oxidase B inhibitor, a glutamate receptor antagonist, a AMPA receptor agonist, a nerve growth factor stimulant, a HMG-CoA reductase inhibitor, a neurotrophic agent, a muscarinic M1 receptor agonist, a GABA receptor modulator, a PPAR-gamma agonist, a microtubule protein modulator, a calcium channel blocker, an antihypertensive agent, a statin, and any combination thereof. In an exemplary embodiment, a pharmaceutical composition may comprise a kinase inhibitor. Suitable kinase inhibitors may inhibit a thousand-and-one amino acid kinase (TAOK), CDK, GSK-3β, MARK, CDK5, or Fyn. In another exemplary embodiment, a pharmaceutical composition may comprise a phosphatase activator. As a non-limiting example, a phosphatase activator may increase the activity of protein phosphatase 2A. In some embodiments the treatment is a pharmaceutical composition comprising a tau targeting therapy, including but not limited to active pharmaceutical ingredients that alter tau phosphorylation patterns, antagonize tau aggregation, or increase clearance of pathological tau isoforms and/or aggregates. In some embodiments, the treatment is an anti-Aβ antibody, an anti-tau antibody, an anti-TREM2 antibody, a TREM2 agonist, a gamma-secretase inhibitor, a beta-secretase inhibitor, a kinase inhibitor, a phosphatase activator, a vaccine, or a tau protein aggregation inhibitor. In one embodiment, a decrease in the pT181/T181 value relative to a healthy control indicates the subject be treated with a sporadic FTD therapy or tau therapy.

In each of the above embodiments, a pharmaceutical composition may comprise an imaging agent. Non-limiting examples of imaging agents include functional imaging agents (e.g. fluorodeoxyglucose, etc.) and molecular imaging agents (e.g., Pittsburgh compound B, florbetaben, florbetapir, flutemetamol, radionuclide-labeled antibodies, etc.)

In each of the above embodiments, the methods may further include quantifying one or more of pT205/T205, pT208/T208, pT111/T111, pT153/T153, or any combination thereof.

### IV. Clinical Trials

Another aspect of the present disclosure is a method for selecting a subject into a clinical trial, in particular a clinical trial for an Aβ or tau therapy, provided all other criteria for the clinical trial have been met. In one embodiment, a method for a method for selecting a subject into a clinical trial may comprise (a) providing a processed CSF or blood sample obtained from a subject, wherein the CSF or blood sample is enriched for one or more ptau and Aβ species; (b) quantifying, in the processed sample, pT217/T217 value and Aβ 42/40 value; and (c) selecting the subject into a clinical trial for MAPT R406W tauopathy when pT217/T217 value is increased and Aβ 42/40 value is about the same as a healthy control population and the subject is without brain amyloid plaques as measured by PET imaging. In another embodiment, a method for a method for selecting a subject into a clinical trial may comprise (a) providing a processed CSF or blood sample obtained from a subject, wherein the CSF or blood sample is enriched for one or more ptau and Aβ species; (b) quantifying, in the processed sample, pT217/T217 value and Aβ 42/40 value; and (c) excluding the subject into a clinical trial for AD (or AD therapy) when pT217/T217 value is increased and Aβ 42/40 value is about the same as a healthy control population and the subject is without brain amyloid plaques as measured by PET imaging.

In another embodiment, a method for selecting a subject into a clinical trial, in particular a clinical trial for an Aβ or tau therapy, provided all other criteria for the clinical trial have been met. In one embodiment, a method for a method for selecting a subject into a clinical trial may comprise (a) providing a processed CSF or blood sample obtained from a subject, wherein the CSF or blood sample is enriched for one or more ptau and Aβ species; (b) quantifying, in the processed sample, a composite pT217/T217 × Aβ 42/40 value; and (c) selecting the subject into a clinical trial for MAPT R406W tauopathy when the composite pT217/T217 × Aβ 42/40 value is increased relative to a healthy control population. In another embodiment, a method for a method for selecting a subject into a clinical trial may comprise (a) providing a processed CSF or blood sample obtained from a subject, wherein the CSF or blood sample is enriched for one or more ptau and Aβ species; (b) quantifying, in the processed sample, a composite pT217/T217 × Aβ 42/40 value; and (c) excluding the subject into a clinical trial for AD when the composite pT217/T217 × Aβ 42/40 value is increased relative to an AD population.

In another embodiment, a method for selecting a subject into a clinical trial, in particular a clinical trial for an Aβ or tau therapy, provided all other criteria for the clinical trial have been met. In one embodiment, a method for a method for selecting a subject into a clinical trial may comprise (a) providing a processed CSF or blood sample obtained from a subject, wherein the CSF or blood sample is enriched for one or more ptau species; (b) quantifying, in the processed sample, a pT181/T181 value; and (c) selecting the subject into a clinical trial for a sporadic FTD therapy when the pT181/T181 value is decreased relative to a healthy control population. In another embodiment, a method for a method for selecting a subject into a clinical trial may comprise (a) providing a processed CSF or blood sample obtained from a subject, wherein the CSF or blood sample is enriched for one or more ptau species; (b) quantifying, in the processed sample, a pT181/T181 value; and (c) excluding the subject into a clinical trial for AD when the composite pT181/T181 value is decreased relative to an AD population. The phrase “a control population without brain amyloid plaques as measured by PET imaging” is defined in Section III.

Alternatively or in addition to using a measurement of site-specific tau phosphorylation, optionally with a measurement of total tau, in any of the above embodiments, a ratio calculated from the measured phosphorylation level(s), or a ratio calculated from the measured phosphorylation level(s) and total tau, may be used. A ratio calculated from the measured phosphorylation level(s) may be a ratio between pT181 and pT205, pT217 and pT205, or pT181 and pT217. A ratio calculated from the measured phosphorylation level(s) and total tau may be a ratio between pT181 and total tau, p-T205 and total tau, or pT217 and total tau. Mathematical operations other than a ratio may also be used. For instance, the examples use site-specific tau phosphorylation values in various statistical models (e.g., linear regressions, LME curves, LOESS curves, etc.) in conjunction with other known biomarkers (e.g. APOE ε4 status, age, sex, cognitive test scores, functional test scores, etc.).

The design of clinical trials for AD and FTD therapies can be greatly aided by the methods disclosed herein. Many clinical trials are designed to test the efficacy of imaging agents or therapeutic agents that target a specific pathophysiological change which occurs prior to the onset of AD symptoms. As discussed above in Section III, the efficacy of these various agents can be improved by administering the agents to subjects that have certain site-specific tau phosphorylation levels, as measured by methods disclosed herein and illustrated. Similarly, clinical trials selecting subjects with symptoms of Aβ pathology or tau only pathology would also benefit from being able to accurately discriminate an enrollee’s pathology in order to determine if efficacy is associated with a particular disease state. Accordingly, measuring tau phosphorylation levels as described herein prior to selecting a subject in a clinical trial, in particular into a treatment arm of a clinical trial, may result in smaller trials and/or improved outcomes. In some instances, methods described herein may be developed and used as a companion diagnostic for a therapeutic agent.

In each of the above embodiments, a subject may be enrolled into a treatment arm of the clinical trial. The “treatment” is defined above. Subjects enrolled in the treatment arm of a clinical trial may be administered a pharmaceutical composition. In some embodiments, a pharmaceutical composition may comprise an imaging agent. Non-limiting examples of imaging agents include functional imaging agents (e.g. fluorodeoxyglucose, etc.) and molecular imaging agents (e.g., Pittsburgh compound B, florbetaben, florbetapir, flutemetamol, radionuclide-labeled antibodies, etc.). Alternatively, a pharmaceutical composition may comprise an active pharmaceutical ingredient. Non-limiting examples of active pharmaceutical ingredients include cholinesterase inhibitors, N-methyl D-aspartate (NMDA) antagonists, antidepressants (e.g., selective serotonin reuptake inhibitors, atypical antidepressants, aminoketones, selective serotonin and norepinephrine reuptake inhibitors, tricyclic antidepressants, etc.), gamma-secretase inhibitors, beta-secretase inhibitors, anti-Aβ antibodies (including antigen-binding fragments, variants, or derivatives thereof), anti-tau antibodies (including antigen- binding fragments, variants, or derivatives thereof), stem cells, dietary supplements (e.g. lithium water, omega-3 fatty acids with lipoic acid, long chain triglycerides, genistein, resveratrol, curcumin, and grape seed extract, etc.), antagonists of the serotonin receptor 6, p38alpha MAPK inhibitors, recombinant granulocyte macrophage colony-stimulating factor, passive immunotherapies, active vaccines (e.g. CAD106, AF20513, etc. ), tau protein aggregation inhibitors (e.g. TRx0237, methylthionimium chloride, etc.), therapies to improve blood sugar control (e.g., insulin, exenatide, liraglutide pioglitazone, etc.), anti-inflammatory agents, phosphodiesterase 9A inhibitors, sigma-1 receptor agonists, kinase inhibitors, phosphatase activators, phosphatase inhibitors, angiotensin receptor blockers, CB1 and/or CB2 endocannabinoid receptor partial agonists, β-2 adrenergic receptor agonists, nicotinic acetylcholine receptor agonists, 5-HT2A inverse agonists, alpha-2c adrenergic receptor antagonists, 5-HT 1A and 1D receptor agonists, Glutaminyl-peptide cyclotransferase inhibitors, selective inhibitors of APP production, monoamine oxidase B inhibitors, glutamate receptor antagonists, AMPA receptor agonists, nerve growth factor stimulants, HMG-CoA reductase inhibitors, neurotrophic agents, muscarinic M1 receptor agonists, GABA receptor modulators, PPAR-gamma agonists, microtubule protein modulators, calcium channel blockers, antihypertensive agents, statins, and any combination thereof. In an exemplary embodiment, a pharmaceutical composition may comprise a kinase inhibitor. Suitable kinase inhibitors may inhibit a thousand-and-one amino acid kinase (TAOK), CDK, GSK-3β, MARK, CDK5, or Fyn. In another exemplary embodiment, a pharmaceutical composition may comprise a phosphatase activator. As a non-limiting example, a phosphatase activator may increase the activity of protein phosphatase 2A.

In each of the above embodiments, a subject may or may not be symptomatic. An “asymptomatic subject,” as used herein, refers to a subject that does not show any signs or symptoms of a tauopathy. Alternatively, a subject may exhibit signs or symptoms (e.g., memory loss, misplacing things, changes in mood or behavior, etc.,) but not show sufficient cognitive or functional impairment for a clinical diagnosis. A symptomatic or an asymptomatic subject may have Aβ amyloidosis; however, prior knowledge of Aβ amyloidosis is not a requisite for treatment. In still further embodiments, a subject may have AD. In any of the aforementioned embodiments, a subject may carry one of the gene mutations known to cause an inherited tauopathy. In alternative embodiments, a subject may not carry a gene mutation known to cause an inherited tauopathy.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

### EXAMPLES

The following examples illustrate various iterations of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

### Example 1 - MAPT R406W Increases Tau T217 Phosphorylation in Absence Of Amyloid Pathology

Tau hyperphosphorylation at threonine 217 (pT217) in cerebrospinal fluid (CSF) has recently been linked to early amyloidosis and could serve as a highly sensitive biomarker for Alzheimer’s disease (AD). However, it remains unclear whether other tauopathies induce pT217 modifications. To determine if pT217 modification is specific to AD, CSF pT217 was measured in AD and other tauopathies.

Using immunoprecipitation and mass spectrometry methods, CSF T217 phosphorylation occupancy (pT217/T217) and amyloid-beta (Aβ) 42/40 ratio was compared in cognitively normal individuals and those with symptomatic AD, progressive supranuclear palsy, corticobasal syndrome, and sporadic and familial frontotemporal dementia.

Individuals with AD had high CSF pT217/T217 and low Aβ42/40. In contrast, cognitively normal individuals and the majority of those with 4R tauopathies had low CSF pT217/T217 and normal Aβ 42/40. We identified a subgroup of individuals with increased CSF pT217/T217 and normal Aβ 42/40 ratio, most of whom were MAPT R406W mutation carriers. Diagnostic accuracies of CSF Aβ 42/40 and CSF pT217/T217, alone and in combination were compared. We show that CSF pT217/T217 × CSF Aβ 42/40 is a sensitive composite biomarker that can separate MAPT R406W carriers from cognitively normal individuals and those with other tauopathies.

MAPT R406W is a tau mutation that leads to 3R+4R tauopathy similar to AD, but without amyloid neuropathology. Thus, the present example provides that change in CSF pT217/T217 ratio is not specific to AD and can reflect common downstream tau pathophysiology common to 3R+4R tauopathies.

Alzheimer’s disease (AD) is characterized by the plaque deposition of amyloid-beta 42 peptide (Aβ 42) and aggregation of hyperphosphorylated tau in neurofibrillary tangles, neuritic plaques, and neuropil threads in the brain. Concomitant decrease in Aβ 42/40 ratio and increase of phosphorylated tau (ptau) species in the cerebrospinal fluid (CSF) have been used as biomarkers for AD amyloidosis and as surrogates of AD tau neuropathology, respectively. Increasing evidence suggests that either CSF tau phosphorylation at threonine 217 measured as absolute concentration (pT217) or as phosphorylation occupancy (pT217/T217) is a specific and more sensitive biomarker for AD, outperforming the well-established measure of CSF ptau level at threonine 181 (pT181). CSF pT181 level increase is strongly associated with the increase in total CSF tau concentration and is assumed to reflect tau neuropathology. However, CSF pT217 and pT217/T217 more strongly correlate with amyloid neuropathology measured by amyloid Pittsburgh compound B (PiB)-positron emission tomography (PET) imaging than total CSF tau. Moreover, pT217 can predict the beginning of AD clinical symptoms in families with AD mutations better than pT181. Thus, it remains unclear if hyperphosphorylation at T217 is an early and direct consequence of amyloidosis or whether it is a downstream marker of tau pathology. In this context, it is also unclear if CSF pT217/T217 changes in primary tauopathies in the absence of amyloid pathology.

Other neurodegenerative dementing illnesses associated with tau neuropathology, including progressive supranuclear palsy (PSP), corticobasal syndrome (CBS), and behavioral variant frontotemporal dementia (bvFTD), currently have no CSF or imaging biomarkers. Diagnosis primarily depends on clinical assessment, which may be confirmed after brain autopsy. The lack of reliable in vivo biomarkers challenges accurate clinical diagnoses, with implications for the design and implementation of clinical trials. Previous studies, mostly measuring absolute CSF pT181 concentrations using immunoassays, suggested that increases in pT181 are specific to AD. However, global change in CSF tau isoforms concentration may contribute to pT181 absolute concentration without relative change in pT181 phosphorylation. pT181 phosphorylation occupancy measured as pT181/T181 ratio is essential to fully interpret the changes in CSF pT181. Furthermore, some recent studies using immunoassays and mass spectrometry (MS) showed that an increase in CSF pT217 concentration is specific to AD and not observed in other neurodegenerative diseases. However, previous studies often do not take into account amyloid co-neuropathology that frequently increases with age and in many neurodegenerative diseases.

In order to evaluate the effect of tau phosphorylation in non-AD tauopathies, including PSP, CBS, and bvFTD, CSF ptau and CSF Aβ were measured using sequential immunoprecipitation (IP) and MS. CSF pT217/T217 and pT181/T181 ratios were calculated to differentiate tau phosphorylation changes from CSF total tau variation. CSF Aβ 42/40 ratio was calculated within the same participant and used as a surrogate for amyloid neuropathology. A cohort of cognitively normal age-matched controls (AMC) and individuals with symptomatic AD, PSP, CBS, and sporadic and familial FTD were analyzed. The correlation between CSF ptau and CSF Aβ 42/40 ratios were assessed in each disease group and evaluated diagnostic relevance of CSF ptau alone and in combination with CSF Aβ 42/40 to assess their ability to discriminate individuals with symptomatic AD from those with other neurodegenerative dementing illnesses.

### Results

Participants and study workflow: Participants’ demographics and clinical characteristics are summarized in Table 1. For the purpose of the analyses, study cohorts were divided into several groups. The “AD” group (n = 80) included patients with amnestic-predominant clinically “typical” AD (n = 66) and those with focal variants (n = 14). bvFTD MAPT R406W mutation carriers (n = 5) were grouped as “R406W.” All neurodegenerative diseases other than AD, and MAPT R406W mutation carriers were grouped as “4R tauopathy” (n = 74). This group included individuals with PSP (n = 16), CBS (n = 15), CBS-PSP continuum (n = 7), sporadic bvFTD (n = 28), and bvFTD MAPT P301L mutation carriers (n = 3), which were primarily 4R tauopathies with 4R tau as primary isoform in the brain aggregates. Note that sporadic bvFTD was listed under “4R tauopathy” group; however, they were pathologically unconfirmed and might contain FTLD-tau, FTLD-TDP43, FTLD-FUS, and small number of 3R tauopathy such as Pick’s disease. One of these participants was later found to have C9orf72 mutation. Cognitively normal controls was named “Control” (n = 98) and included AMC (n = 64), YNC (n = 26), and brain tumor patients (n = 8) who were cognitively normal.

TABLE 1

Participants’ demographics and clinical characteristics

| Groups | AD (n = 80) | | 4R tauopathy¹ (n = 74) | | | | |
|---|---|---|---|---|---|---|---|
| Subcategories | AD (n = 66) | AD focal² (n = 14) | PSP (n = 16) | CBS (n = 15) | CBS PSP continuum (n = 7) | bvFTD (N = 36) | |
| | | | | | | Sporadic bvFTD3 (n = 28) | P301L (n = 3) |
| Age ± SEM | 74.5 ± 1.9 | 66.7 ± 2.2 | 71.0 ± 2.6 | 68.6 ± 2.6 | 70.7 ± 1.4 | 62.1 ± 1.3 | 37.2 ± 3.6 |
| Average age | 73.3 ± 0.9 | | 64.5 ± 1.3 | | | | |
| Sex (M/F) | 34/32 | 8-Jun | 7-Sep | 9-Jun | 3-Apr | 16/12 | Mar-00 |
| WashU-A | 41 | 0 | 0 | 0 | 0 | 0 | 0 |
| WashU-B | 11 | 0 | 6 | 3 | 0 | 6 | 3 |
| Montpellier | 14 | 14 | 10 | 12 | 7 | 22 | 0 |

| | | | | | | |
|---|---|---|---|---|---|---|
| Groups | R406W (n = 5) | Control (N = 98) | | | | All (n = 252) |
| Subcategories | R406W (n = 5) | AMC (n = 64) | | YNC (n = 26) | Brain Tumor (n = 8) | Total |
| Age ± SEM | 54.8 ± 6.5 | 73.3 ± 0.8 | 42.4 ± 2.4 | 50.2 ± 2.6 | 66.8 ± 0.8 | |
| Average age | | 63.4 ± 1.6 | | | | |
| Sex (M/F) | 3-Feb | 32/32 | 19-Jul | 4-Apr | 123/129 | |
| WashU-A | 0 | 44 | 0 | 0 | 85 | |
| WashU-B | 5 | 20 | 26 | 8 | 88 | |
| Montpellier | 0 | 0 | 0 | 0 | 79 | |

AD, Alzheimer’s disease; PSP, progressive supranuclear palsy; CBS, corticobasal syndrome; bvFTD, behavioral variant frontotemporal dementia; AMC, age-matched controls; YNC, Young normal controls; CSF, cerebrospinal fluid.
¹ For the purpose of analyses, all neurodegenerative diseases other than AD including PSP, CBS, CBS PSP continuum, sporadic bvFTD, and FTD MAPT P301L mutation carriers, which are primarily 4R tauopathies are grouped as “4R tauopathies.”
² AD focal is defined as individuals with predominant language, behavioral, visuospatial, apraxia phenotype with CSF biomarkers of AD. It is categorized under “AD.”
³ Sporadic bvFTD is listed under “4R tauopathy” group; however, may contain undiagnosed FTD-TPD43, FTD-FUS, and 3R tauopathy cases. N = 1 was later found to have C9orf72 mutation.

Individuals in YNC (42.3 ± 2.4), Brain tumor (50.2 ± 2.6), and MAPT P301L (37.2 ± 3.6) groups were younger than AMC (73.0 ± 0.8) and participants with neurodegenerative diseases including AD (73.3 ± 0.9), CBS (68.6 ± 2.6), CBS PSP continuum (70.7 ± 1.4), PSP (71.0 ± 2.6), and sporadic bvFTD (62.1 ± 1.3) (Table 1).

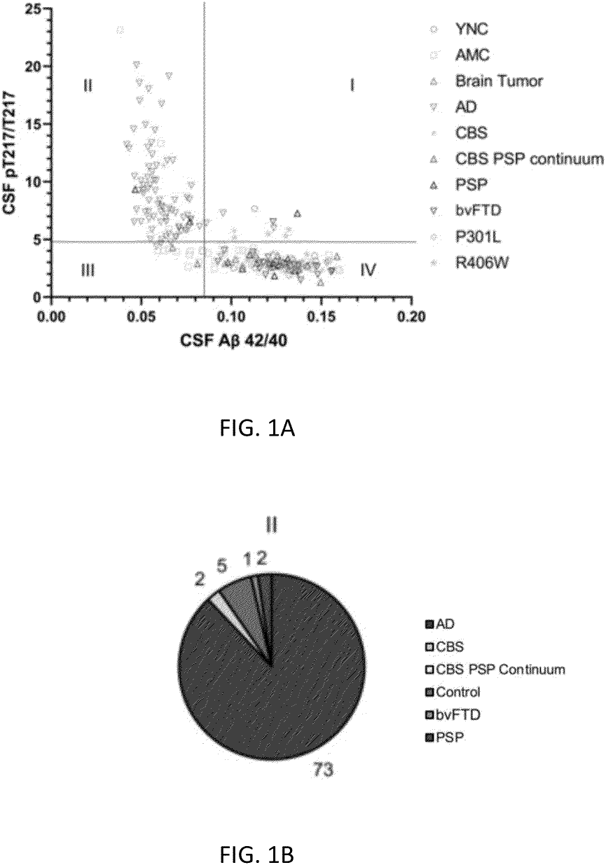

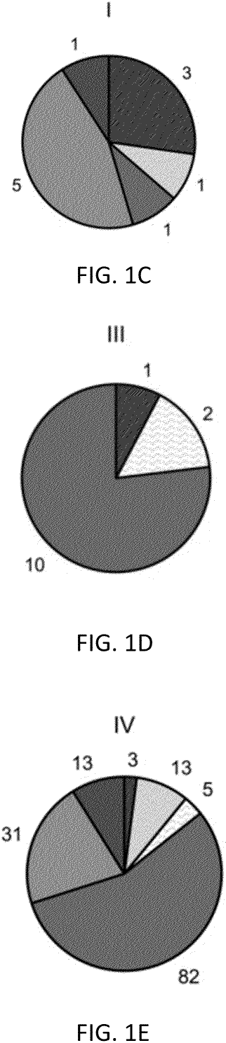

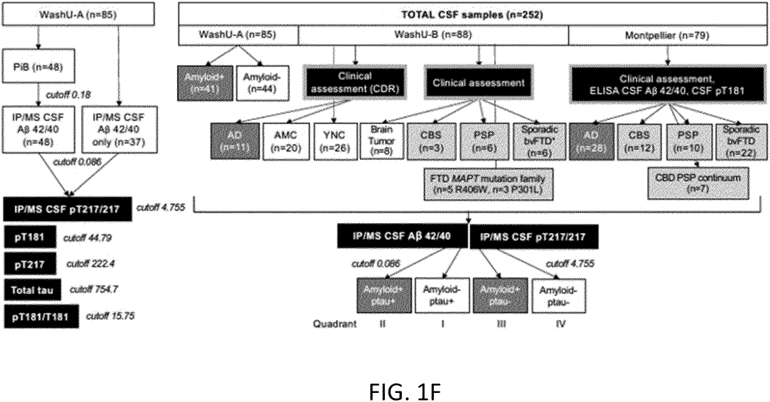

All 252 CSF baseline samples and 8 CSF follow-up samples were measured with sequential IP/MS methods for CSF Aβ 42, Aβ 40, pT217, T217, pT181, and T181 concentrations. CSF Aβ 42/40, pT217/T217, pT181/T181 ratios were calculated. The workflow used to categorize the different clinical groups is described in FIG. 1F.

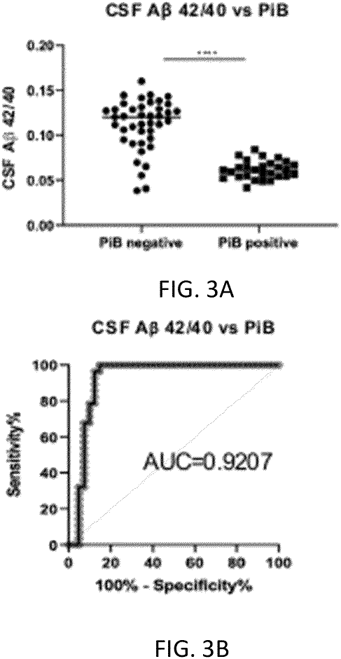

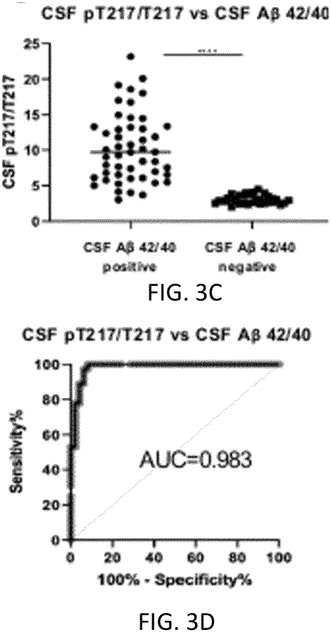

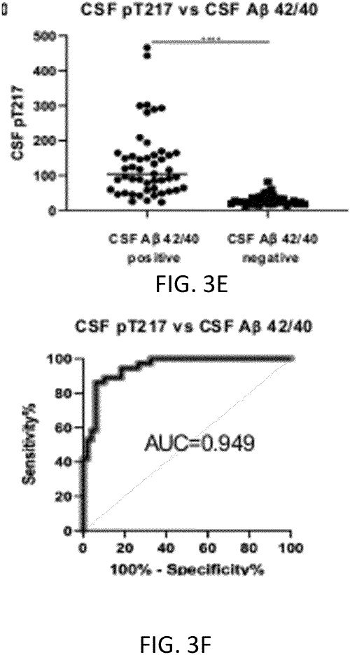

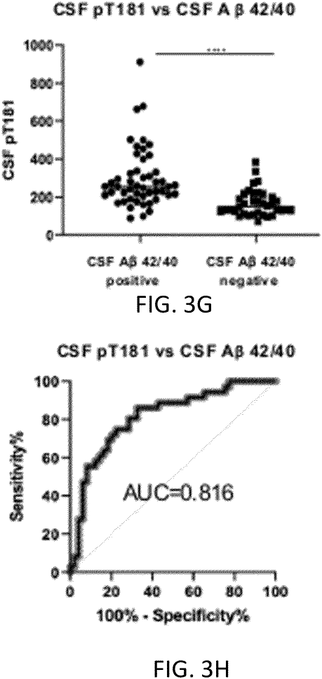

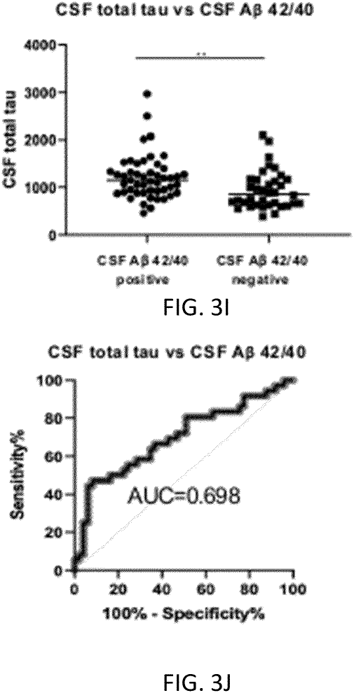

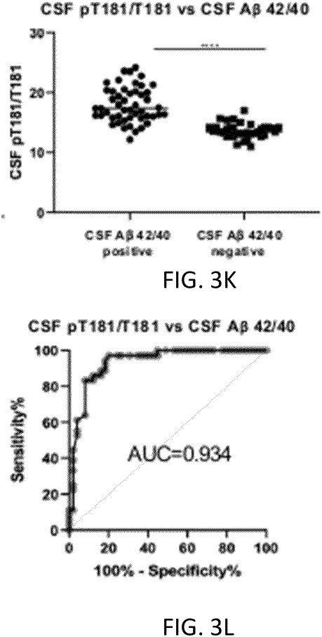

Determining cutoffs for IP/MS CSF Aβ 42/40 and CSF pT217/T217: To define amyloid positivity cutoff for CSF Aβ 42/40 measured by IP/MS, amyloid PiB-PET results were used from 48 participants in the WashU-A cohort (cutoff 0.18; FIG. 1F, Table 1). CSF Aβ 42/40 was significantly decreased in the amyloid-PiB+ cohort (FIG. 3A). A ROC curve analysis was performed and a Youden’s index value of 0.086 was selected as a cutoff for CSF Aβ 42/40 to maximize discrimination between cohorts (area under the curve [AUC] = 0.921, p < 0.0001; FIG. 3B).

To determine ptau abnormality cutoff for CSF pT217/T217, CSF Aβ 42/40 values in amyloid-PiB+ patients were used from the WashU-A cohort (FIG. 1F). Another subset of 37 participants with only MS were added CSF Aβ 42/40 measurements. CSF pT217/T217 was significantly increased in amyloid+ individuals based on PiB-PET and CSF Aβ 42/40 measurements (FIG. 3C). A ROC curve analysis was performed and a Youden’s index value of 4.76 was calculated as a cutoff for CSF pT217/T217 (AUC = 0.983, p < 0.0001; FIG. 3D).

The same ROC analyses were performed for concentrations of CSF pT217, pT181, total tau, and phosphorylation occupancy at T181 (pT181/T181. FIGS. 3E-3L). AUC for each of these biomarkers was 0.949 (pT217), 0.816 (pT181), 0.698 (total tau), and 0.934 (pT181/T181), respectively, supporting the previous finding that CSF pT217/T217 is a superior discriminative AD biomarker.

Association between IP/MS CSF Aβ 42/40 and CSF pT217/T217: To evaluate the relationship between IP/MS CSF Aβ 42/40 and pT217/T217, both ratios were plotted for each of the 255 participants. Based on the calculated 0.086 and 4.8 cutoffs for CSF Aβ 42/40 and CSF pT217/T217, respectively, quadrants were defined as follows: I (amyloid-, ptau+), II (amyloid+, ptau+), III (amyloid+, ptau-), and IV (amyloid-, ptau-) (FIGS. 1A-1E, FIG. 3B).

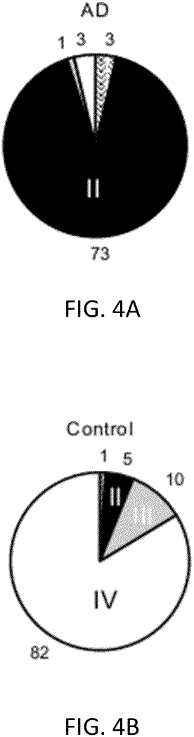

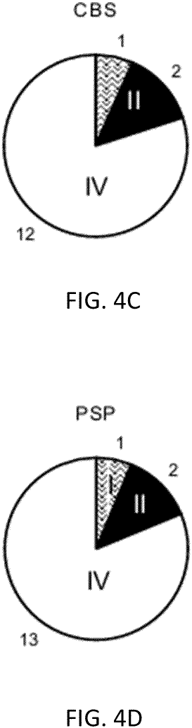

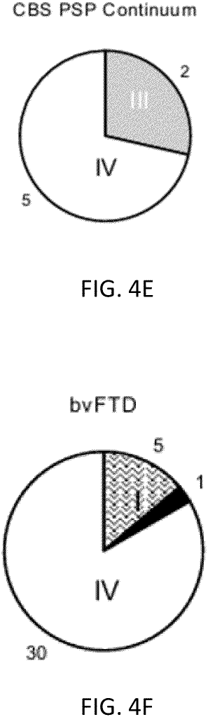

In quadrant II (amyloid+, ptau+), 88% (73/83) of individuals were clinically identified as AD (FIG. 1B). Overall, 91% (73/80) of individuals with AD were plotted in II (FIG. 4A). Seven participants clinically identified as AD were divided into quadrant I (n = 3), III (n = 1), and IV (n = 3, FIGS. 1B-1D). A subset of Controls (AMC [n = 5], CBS [n = 2], PSP [n = 2] and bvFTD [n = 1]) were also assigned to quadrant II (FIGS. 1A and 1B).

Eighty four percent (82/98) of controls were plotted in IV and 55% (82/150) of individuals in IV were controls (FIG. 1E, FIG. 4B). Eighty percent (12/15) of CBS and 81% (13/16) of PSP were also in IV (FIGS. 4C and 4D) as well as 71% (5/7) of the CBS PSP continuum.

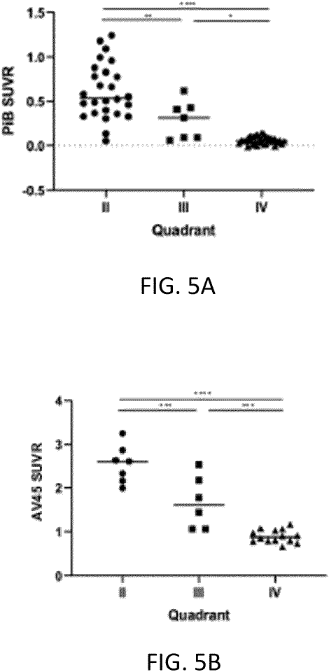

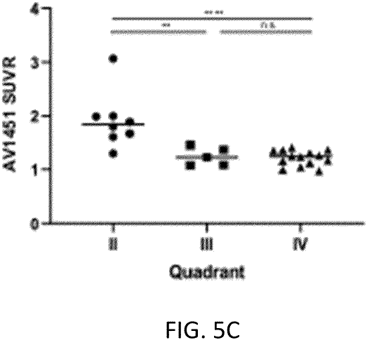

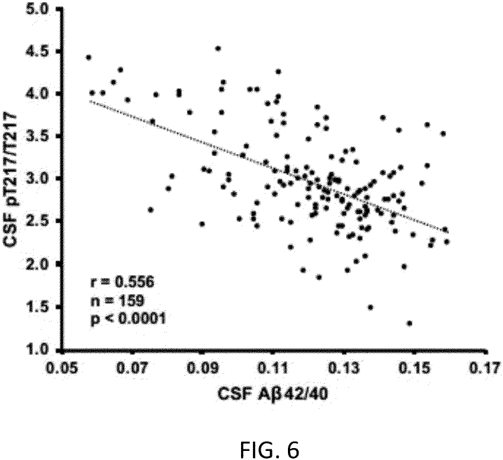

CSF Aβ 42/40 and CSF pT217/T217 were negatively associated and displayed an L-shaped curve (FIGS. 1). To better understand the dynamic association between CSF Aβ 42/40 and CSF pT217/T217 profile in the context of AD continuum, amyloid burden was assessed and measured with PiB-PET and AV45-PET imaging and tau aggregation by AV1451-PET imaging in a subset of participants (FIGS. 5). Aβ aggregation measured by both PET tracers gradually and significantly increased from quadrant IV, III to II (FIGS. 5A and 5B). All the Controls in quadrant III were AMC (FIG. 1A) and none belonged to the YNC (age <64). However, within AMC, there was no significant difference in age in quadrant III (74.8 ± 2.4) and quadrant IV (72.4 ± 0.6). These suggest that individuals in quadrant III with amyloid positivity may be defined as presymptomatic AD without abnormal tau phosphorylation. In contrast, tau aggregation measured by AV1451-PET only increased in quadrant II (FIG. 5C), supporting CSF Aβ 42/40 and amyloid PET change before CSF pT217/T217 and tau PET. Importantly, a significant negative correlation between CSF Aβ 42/40 and CSF pT217/T217 was observed in quadrants III and IV (ptau-) including most controls, PSP, CBS, and bvFTD (FIG. 6).

MAPT R406W carriers have increased pT217/T217 ratio without amyloid pathology: Quadrant I (amyloid-, ptau+) was populated by individuals with bvFTD, PSP, AD, or CBS (FIG. 1C). Interestingly, 45% (5/11) were bvFTD, and all but one (80%, 4/5) were MAPT R406W mutation carriers (FIG. 4F, Table 2). All (5/5) MAPT R406W mutation carriers analyzed in this study were amyloid negative (quadrant I and IV), supporting the absence of amyloid neuropathology in MAPT R406W carriers (Table 2). Only one out of five MAPT R406W mutation carriers who were in their 40s and asymptomatic throughout the study was CSF pT217/T217 negative (quadrant IV) at both baseline and follow-up visit; all other MAPT R406W mutation carriers were CSF pT217/T217 positive (quadrant I) regardless of their pre/symptomatic status. One participant (#5) who was asymptomatic at baseline and developed dementia in follow-up visit 4 years later had CSF pT217/T217 just below the threshold at baseline (quadrant IV), but CSF pT217/T217 increased in the follow-up (quadrant I), suggesting that the longitudinal changes in this biomarker could reflect disease progression. In comparison, five other participants with follow-up visits within 1 year (1 PSP in quadrant I, 2 CBS, and 2 AMC in quadrant IV) remained in the same quadrant between baseline and follow-up. These results suggest that increasing age and emergence of symptoms associate with increases in CSF pT217/T217 in MAPT R406W mutation carriers.

TABLE 2

Demographics and summary of biomarker values for MAPT R406W mutation carriers

| Participent | Quadrant | Symptomatic/Asymptomatic | Age | Sex | CSF Aβ 42/40 | CSF pT217/T217 | CSF pT217/T217 × CSF Aβ 42/40 |
|---|---|---|---|---|---|---|---|
| | | | | | (cutoff = 0.086) | (cutoff = 4.76] | (cutoff for R4O6W versus control = 0.50) |
| #1 | IV | No dementia | 40 | M | 0.142 | 3.69 | 0.524 |
| | IV | No dementia | 41 | | 0.152 | 3.75 | 0.572 |
| #2 | I | No dementia | 4D | M | 0.13 | 5.28 | 0.658 |
| #3 | I | No dementia | 52 | F | 0.127 | 5.3 | 0.671 |
| | I | No dementia | 55 | | 0.132 | 5.86 | 0.774 |
| #4 | I | Symptomatic | 64 | | 0.121 | 5.47 | 0.653 |
| #5 | IV | Asymptomatic, other primary | 69 | F | 0.108 | 4.73 | 0.513 |
| | I | Symptomatic | 73 | | 0.102 | 5.14 | 0.522 |
| Average (age <50) | 11.2 IV | No dementia | 40.3 ± 0.40 | - | 0.142 ± 0.0064 | 4.2 ± 0.52 | 0.595 ± 0.049 |
| Average (age >50) | 41.1 IV | No dementia, Symptomatic. other primary | 63.0 ± 4.1 | - | 0.118 ± 0.0056 | 5.3 ± 0.19 | 0.628 ± 0.049 |
| Average all | -- | -- | 54.5 ± 4.8 | - | 0.127 ± 0.0059 | 4.9 ± 0.28 | 0.615 ± 0.049 |

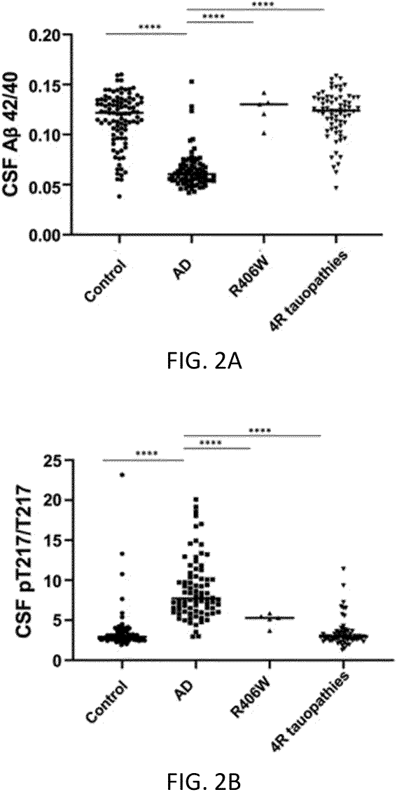

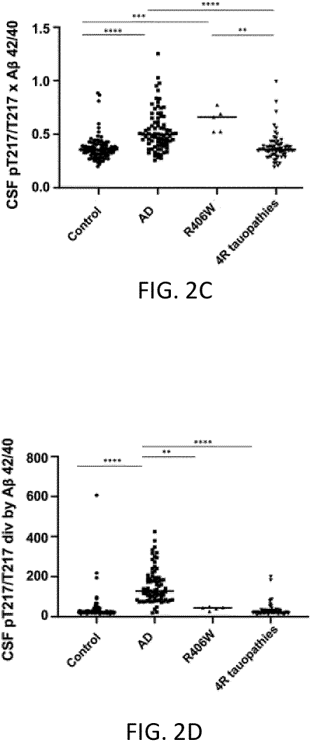

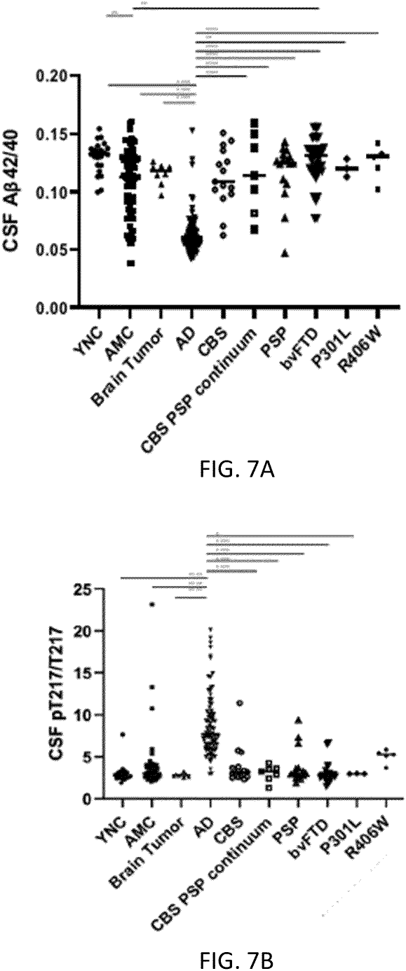

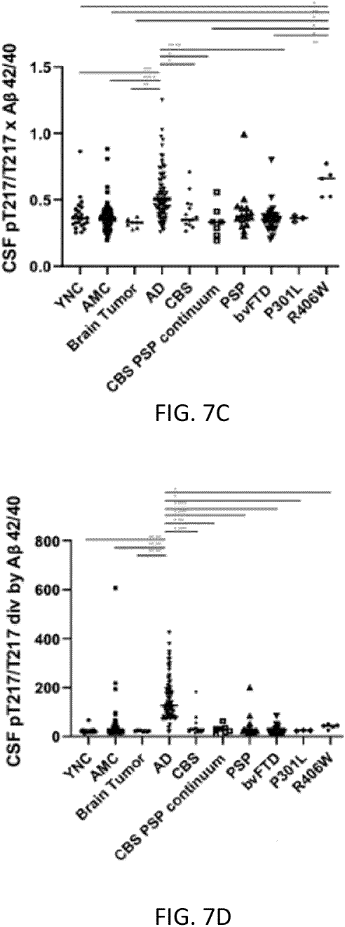

Diagnostic values of IP/MS CSF Aβ 42/40, pT217/T217, and composite biomarkers in AD and MAPT R406W mutation carriers: Next, the diagnostic performance of IP/MS CSF Aβ 42/40 and CSF pT217/T217 was compared, with composite biomarkers consisting of pT217/T217 multiplied by CSF Aβ 42/40 and CSF pT217/T217 divided by Aβ 42/40 (FIGS. 2, Table 3, FIGS. 7). For this analysis, the four clinical groups previously defined (“AD,” “R406W,” “4R tauopathy,” and “Control”) were compared. CSF Aβ 42/40 ratio and CSF pT217/T217 used alone only separate AD from the other three groups (FIGS. 2A and 2B). However, the R406W group had significantly increased CSF Aβ 42/40 × pT217/T217 composite biomarker compared to what was observed in the Control and 4R tauopathy groups (FIG. 2C). This composite biomarker demonstrated excellent ability to separate the R406W group from the 4R tauopathy (AUC = 0.948) and Control groups (AUC = 0.961). When this composite biomarker was used, 100% of MAPT R406W mutation carriers were above the cutoff of 0.50 for R406W versus control (Table 3). CSF pT217/T217 divided by CSF Aβ 42/40 performed similarly to CSF Aβ 42/40 and CSF pT2117/T217 alone, and could not distinguish R406W from other groups (FIG. 2D, Table 3).

TABLE 3

Diagnostic accuracy of combinations of CSF Aβ 42/40 and CSF pT217T217 biomarkers for AD and FTD-MAPT R406W

| Test | Diagnostic groups | n per group | AUC | 95% Cl | P value | Sensitivity % | Specificity % | Cutoff |
|---|---|---|---|---|---|---|---|---|
| CSF Aβ 42/40 | AD vs R406W | 80 vs 8 | 0.977 | 0.9464 to 1.000 | <0.0001 | 100.0 | 96.3 | >0.09853 |
| | AD vs 4R tauopathy | 80 vs 74 | 0.949 | 0.9088 to 0.9885 | <0.0001 | 94.6 | 90.0 | >0.07678 |
| | Control vs AD | 100 vs 80 | 0.926 | 0.8830 to 0.9687 | <0.0001 | 90.0 | 90.0 | <0.07669 |
| | Control vs R406W | 100 vs 8 | 0.599 | 0.4197 to 0.7778 | 0.3541 | 75.0 | 46.0 | >0.1204 |
| | Control vs 4R tauopathy | 100 vs 74 | 0.556 | 0.4701 to 0.6421 | 0.2065 | 59.5 | 56.0 | >0.1231 |
| | 4R tauopathy vs R406W | 74 vs 8 | 0.554 | 0.3578 to 0.7504 | 0.617 | 55.4 | 62.5 | <0.1265 |
| CSF pT217/T21 7 | Control vs AD | 100 vs 80 | 0.952 | 0.9163 to 0.9868 | <0.0001 | 96.3 | 93.0 | > 4.355 |
| | AD vs 4R tauopathy | 80 vs 74 | 0.947 | 0.9114 to 0.9818 | <0.0001 | 88.3 | 96.3 | < 4.335 |
| | Control vs R406W | 100 vs 8 | 0.898 | 0.8256 to 0.9706 | 0.0002 | 100.0 | 76.0 | > 3.675 |
| | AD vs R406W | 80 vs 8 | 0.894 | 0.8253 to 0.9622 | 0.0003 | 100.0 | 80.0 | < 5.915 |
| | 4R tauopathy vs R406W | 74 vs 8 | 0.872 | 0.7986 to 0.9449 | 0.0006 | 81.81 | 100.0 | < 3.670 |
| | Control vs 4R tauopathy | 100 vs 74 | 0.539 | 0.4518 to 0.6253 | 0.3795 | 18.2 | 94.0 | < 2.345 |
| CSF pT217/T21 7 x CSFAβ 42/40 | Control vs R406W | 100 vs 8 | 0.960 | 0.9235 to 0.9965 | <0.0001 | 100.0 | 94.0 | > 0.4989 |
| | 4R tauopathy vs R406W | 74 vs 8 | 0.934 | 0.8802 to 0.9880 | <0.0001 | 90.51 | 100.0 | < 0.5078 |
| | Control vs AD | 100 vs 80 | 0.813 | 0.7471 to 0.8791 | <0.0001 | 71.3 | 86.0 | > 0.4295 |
| | AD vs 4R tauopathy | 80 vs 74 | 0.783 | 0.7080 to 0.8583 | <0.0001 | 85.1 | 68.8 | < 0.4356 |
| | AD vs R406W | 80 vs 8 | 0.719 | 0.6069 to 0.8306 | 0.0421 | 100.0 | 60.0 | >0.5100 |
| | Control vs 4R tauopathy | 100 vs 74 | 0.534 | 0.4474 to 0.6213 | 0.4394 | 75.7 | 36.0 | >0.3316 |
| CSF pT217/T21 7 divided by CSF Aβ 42/40 | AD vs R406W | 80 vs 8 | 0.964 | 0.9249 to 1.000 | <0.0001 | 100.0 | 93.8 | < 73.04 |
| | AD vs 4R tauopathy | 80 vs 74 | 0.954 | 0.9182 to 0.9906 | <0.0001 | 93.2 | 95.0 | < 67.60 |
| | Control vs AD | 100 vs 80 | 0.950 | 0.9129 to 0.9874 | <0.0001 | 95.0 | 95.0 | > 69.77 |
| | 4R tauopathy vs R406W | 74 vs 8 | 0.797 | 0.6868 to 0.9078 | 0.006 | 85.1 | 75.0 | < 37.95 |
| | Control vs R406W | 100 vs 8 | 0.771 | 0.6608 to 0.8817 | 0.0109 | 75.0 | 82.0 | > 39.93 |
| | Control vs 4R tauopathy | 100 vs 74 | 0.534 | 0.4469 to 0.6218 | 0.4394 | 85.1 | 24.0 | < 35.43 |

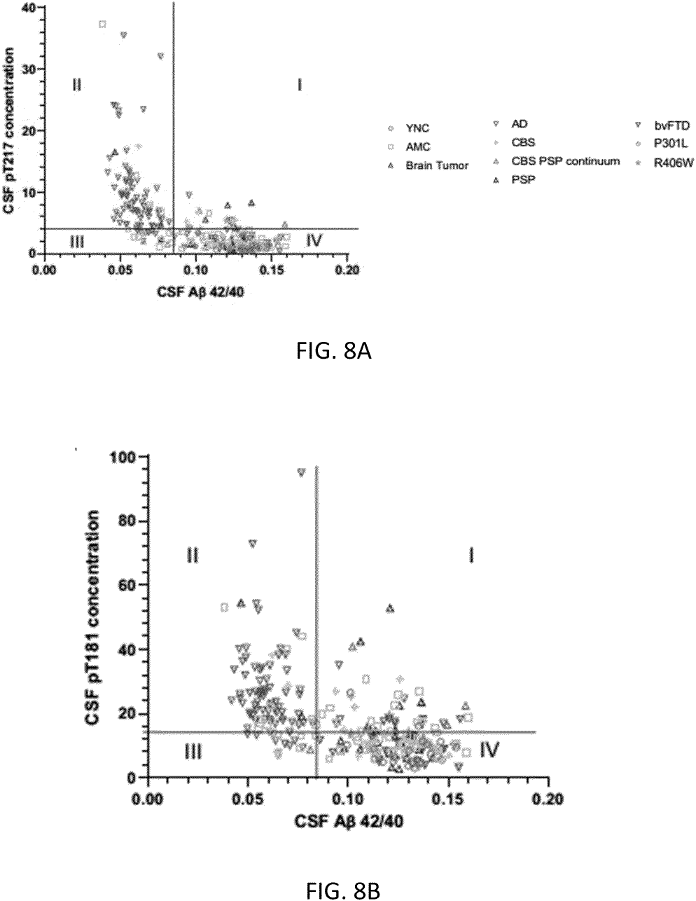

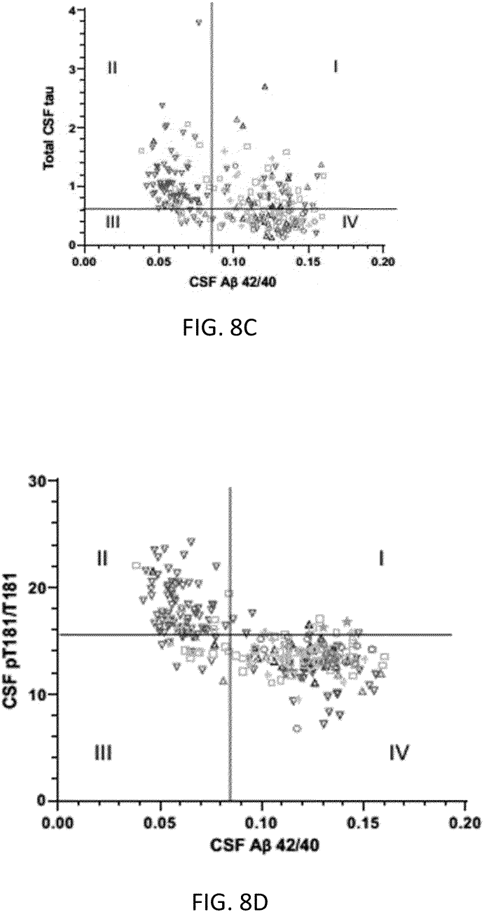

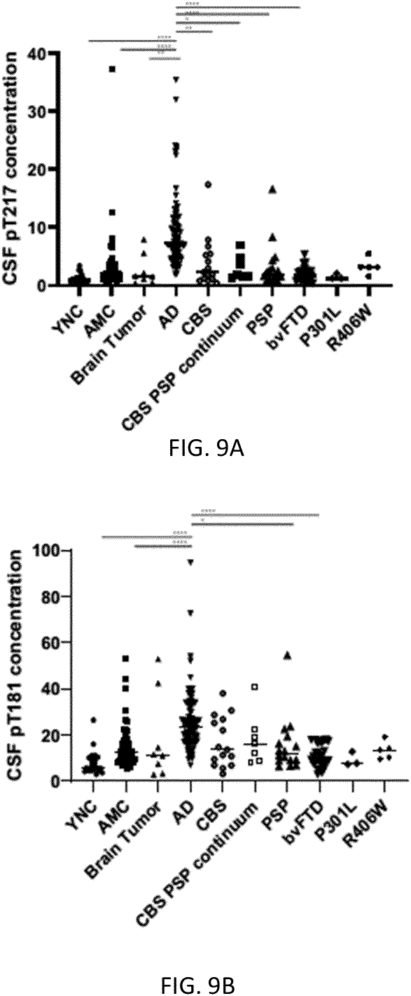

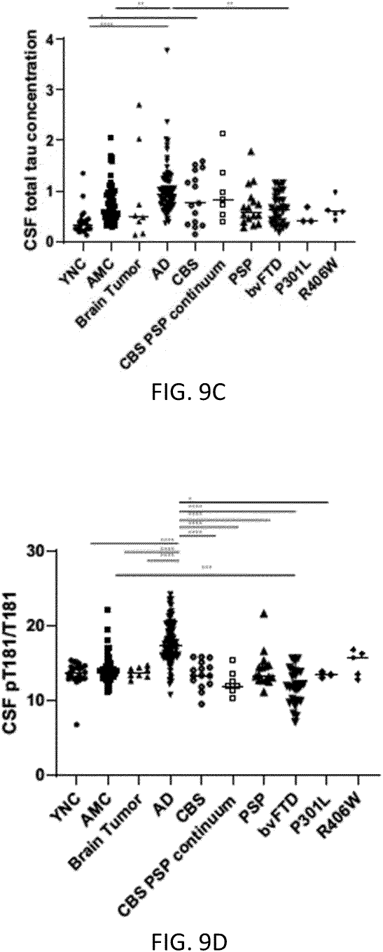

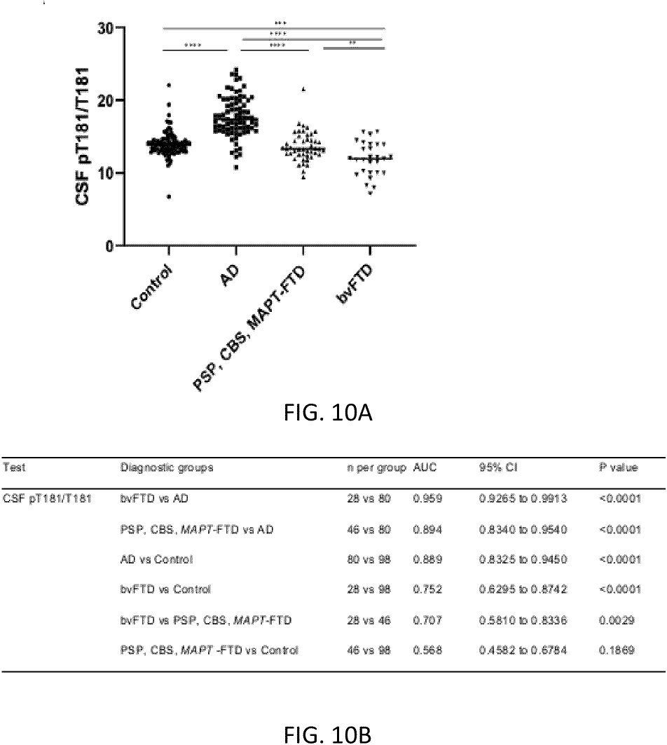

IP/MS CSF total tau and ptau concentrations are not efficient biomarkers for MAPT R406W carriers: Neither CSF pT217, pT181, total tau, nor phosphorylation occupancy at T181 (pT181/T181) were as efficient as the composite biomarker CSF pT217/T217 × CSF Aβ 42/40 at separating MAPT R406W mutation carriers from individuals with other neurodegenerative dementing illnesses (FIGS. 8, FIGS. 9). CSF pT181/T181 was significantly decreased in sporadic bvFTD (including FTLD-tau, FTLD-TDP43, and FTLD-FUS) compared to AD, AMC, and MAPT R406W mutation carriers (FIG. 8D, FIG. 9D). When regrouped, CSF pT181/T181 was able to separate sporadic bvFTD group from AD (AUC = 0.959), Control (AUC = 0.752), and other tauopathies including CBS, PSP, and FTD-MAPT including R406W and P301L (AUC = 0.707. FIGS. 10). However, the specificity and sensitivity of CSF pT181/T181 to separate sporadic bvFTD from other tauopathies and control were not as high as that of CSF pT217/T217 in identifying MAPT R406W mutation carriers.

### Discussion

MAPT R406W mutation carriers have increased pT217/T217 without amyloid pathology: CSF pT217/T217 strongly correlates with amyloid pathology measured by amyloid PET in AD, but it was unproven whether pT217/T217 was a readout for CSF amyloid pathology or tau pathology. This example showed a specific correlation between CSF Aβ 42/40 and CSF pT217/T217 in individuals with symptomatic AD. Even in presymptomatic AD, CSF Aβ 42/40 and CSF pT217/T217 correlate when slight changes in phosphorylation are observed, consistent with previous reports showing a correlation between PiB-PET and CSF pT217/T217. Neither CSF Aβ 42/40 nor CSF pT217/T217 were altered in other tauopathies, including PSP, CBS, and most sporadic and familial FTD. However, it was found that MAPT R406W mutation carriers have increased CSF pT217/T217 independent of amyloid pathology, demonstrating that increased CSF pT217/T217 is, indeed, a biomarker of pathological tau modification common to AD and MAPT R406W associated dementia and that amyloid pathology is not a prerequisite to this modification. This suggests that there is a common tau pathology downstream of AD and MAPT R406W mutation carriers, which results in specific tau phosphorylation changes in the brain, leading to an increase in CSF pT217/T217. Alternatively, two distinct upstream mechanisms, one involving amyloid deposition and the second involving MAPT R406W mutation, could lead to the activation of a similar pathway, ultimately leading to tau hyperphosphorylation and aggregation.

MAPT R406W mutation’s similarity to AD: MAPT R406W mutation-related pathology shares multiple clinical and neuropathological similarities with AD. Unlike other MAPT mutation carriers, MAPT R406W mutation carriers have later ages-at-symptomatic onset, with clinical symptoms including memory loss emerging, on average, in the mid-50s with slow progression. Most pathological MAPT mutations such as P301L are located in or around exon 10 and typically lead to 4R tau isoform aggregation, resulting in 4R tauopathies. In contrast, the MAPT mutations such as R406W and V337M are located in the C-terminus of the tau protein in a domain common to both 3R and 4R tau isoforms, resulting in 3R+4R mixed brain pathologies. The MAPT R406W mutation, like AD, can thus be categorized as a 3R+4R tauopathy and is differentiated from other 4R (CBS, PSP, bvFTD related to MAPT mutations located on exon 10) or 3R (Pick’s disease) tauopathies.

Filament structures in tau aggregates have been recently resolved by cryo-electron microscopy for different tauopathies such as AD and chronic traumatic encephalopathy (CTE) (3R+4R), CBS (4R), and Pick’s disease (3R). Consistent with neuropathological findings, tau domains shared by 3R and 4R isoforms are involved in AD and CTE tau aggregates, while 4R and 3R specific domains are, respectively, involved in corticobasal degeneration and Pick’s disease aggregates. Though no such structural data is available for MAPT R406W, AD, MAPT R406W, and V337M have paired helical filament structures, and AD Tau PET tracers such as AV1451 bind to some extent in presymptomatic MAPT R406W and V337M mutation carriers but not in other tauopathies, suggesting that tau aggregates in these 3R+4R tauopathies have similar characteristics. However, how hyperphosphorylation at T217 contributes to or associates with paired helical filament formation remains to be addressed. Previous studies suggest that CSF T217 is hyperphosphorylated in the early presymptomatic stages of AD, and is detectable more than 20 years before the emergence of clinical symptoms, while tau aggregates detected by PET imaging increase near symptom onset. The present example provides that in 3R+4R tauopathies including MAPT R406W mutation carriers, (1) CSF pT217/T217 becomes abnormal prior to symptom onset when tau paired helical filament formation begins but it is below the detection limit by tau PET imaging followed by evident changes in Tau PET imaging; or, (2) CSF T217 hyperphosphorylation is not directly associated with the formation of neurofibrillary tangles but reflects an abnormal cellular metabolism affecting tau and leading ultimately to tau aggregation.

Composite biomarker of CSF pT217/T217 × CSF Aβ 42/40 serves as a sensitive biomarker for MAPT R406W mutation carriers: Diagnostic values of CSF pT217/T217 and CSF Aβ 42/40 alone and in combination were evaluated. CSF pT217/T217 levels were increased in MAPT R406W mutation carriers. However, the degree of increase was much smaller compared to that of AD and MAPT R406W mutation carriers could not be separated from the Control or 4R tauopathy groups including PSP, CBS, sporadic bvFTD, and FTD-MAPT P301L by CSF pT217/T217 alone (FIGS. 2A, 2B, FIG. 6). Previous studies have indicated an increase of CSF and plasma pT181 concentrations in some cases of MAPT R406W mutation carriers, but the increase was mild. These are consistent with the insufficient sensitivity obtained from this study using only CSF concentrations of pT181, pT217, or phosphorylation occupancies at T181 and T217 (pT181/T181 and pT217/T217) as R406W biomarkers. Through quadrant analysis, it was demonstrated that both CSF pT217/T217 and CSF Aβ 42/40 are necessary to distinguish MAPT R406W mutation carriers with high accuracy. It was also demonstrated that a composite biomarker, CSF pT217/T217 × CSF Aβ 42/40, contained sufficient sensitivity and specificity to distinguish MAPT R406W mutation carriers from the controls and 4R tauopathies (FIG. 2C, FIG. 7C. AUC = 0.934, 0.960, respectively). This is comparable to the high specificity and sensitivity of CSF Aβ 42/40 and CSF pT217/T217 to distinguish AD from the controls (AUC = 0.926, 0.952, respectively). A combination of CSF Aβ 42/40 and pT217/T217 ratios could be used in future trials to select for presymptomatic MAPT R406W mutation carriers and possibly other 3R+4R tauopathies such as V337M. Moreover, longitudinal measures of CSF pT217/T217 could reflect disease progression, suggesting that CSF Aβ 42/40, CSF pT217/T217, and composite biomarkers may serve as new sensitive readouts in drug clinical trials against tauopathies that can assess target engagement in MAPT R406W mutation carriers.

CSF pT181/T181 decreases in sporadic bvFTD: Previous studies using immunoassays showed mixed results in bvFTD, PSP, and CBS patients showing no or mild changes in CSF total tau or pT181. Consistent with multiple reports, the present example did not show significant differences in CSF total tau or CSF pT181 concentrations alone between bvFTD, PSP, CBS, and Control groups. However, by calculating the phosphorylation occupancies within the same participant, it was shown that CSF pT181/T181 significantly decreases in sporadic bvFTD. This may be achieved by normalizing the changes in pT181 by T181, accounting for any physiological increase in pT181 as total tau increases, and individual variabilities such as age, sex, and genotype. Specificity and sensitivity of CSF pT181/T181 biomarker in identifying sporadic bvFTD from Controls or other tauopathies (AUC <0.8) were not as high as a composite biomarker, CSF pT217/T217 × CSF Aβ 42/40, in identifying MAPT mutation carriers (AUC >0.9). This may be due to the heterogeneity of the sporadic bvFTD cohort including FTLD-tau, FTLD-TDP, and FTLD-FUS.

### Methods

Human studies: All studies were approved by the Institutional Review Board at Washington University in St. Louis, MO, USA and the Ethics Committee of the Montpellier University Hospital (CSF-NeuroBANK #DC-2008-417 at the certified NFS 96-900 CHU resource center BB-0033-00031 [http://www.biobanques.eu]). All participants or their delegates consented to the collection and sharing of biofluid samples. Exclusion criteria included contradiction to lumbar punctures (LPs) or lumbar catheters including a bleeding disorder, active anticoagulation, and active infection. Authorization to handle personal data was granted by the French Data Protection Authority (CNIL) under the number 1709743 v0.

AMC and individuals with mild AD were recruited at the Knight Alzheimer Disease Research Center (ADRC) at Washington University School of Medicine as part of Stable Isotope Labeling Kinetics (SILK) studies. AMC are volunteers who were enrolled for research purposes and are cognitively normal. This included two distinct cohorts of symptomatic participants (WashU-A, and WashU-B). Individuals from the WashU-A cohort participated in 36 hr lumbar catheter studies. Individuals from the WashU-B cohort participated in the SILK study that involved five LPs over 4 months. Individuals were diagnosed by clinical assessment and classified according to the Clinical Dementia Rating (CDR). In addition to interviews of patient and collateral source, brain PET imaging data and diagnostic CSF results were reviewed if available. This cohort includes clinical AD patients who did not have biomarkers consistent with AD, who were classified as non-AD dementia. WashU-A cohort was further classified with Amyloid PET positivity based on PiB imaging. Young normal controls (YNC) between the ages of 18-64 without currently diagnosed neurological disorders, were referred from Volunteers for Health at Washington University. Brain tumor patients were referred from Barnes Jewish Hospital. Patients with PSP, CBS, and sporadic bvFTD were referred from affiliated Memory Diagnostic Center and Movement Disorders Clinics. MAPT P301L and R406W mutation carriers were clinically assessed locally at Washington University and referred from the Longitudinal Evaluation of Familial Frontotemporal Dementia Study (LEFFTDS; allftd.org/artfl-lefftds; Site PI NG). Eight participants (1 PSP, 3 MAPT R406W, 2 CBS, and 2 AMC) had repeated LPs and CSF collection.

Montpellier participants were referred from the Memory Resources and Research Center of Montpellier. They were categorized into AD, CBS, PSP, bvFTD, and CBS-PSP continuum based on clinical, neuropsychological, brain imaging, and follow-up assessments. CSF biomarkers of Aβ42, tau, pT181 concentrations were also measured with Enzyme-Linked immunosorbent Assay (ELISA) and Aβ42/40 ratio was calculated. AD was diagnosed according to accepted criteria and based on the ATN classification; all AD participants had at least two abnormal CSF biomarkers. This includes AD focal phenotype which refers to predominant language, behavioral, visuospatial, apraxia phenotype with CSF biomarkers of AD. Some PPA endophenotype AD cases were included in the AD focal phenotype (n = 5). CBS and PSP were diagnosed according to international criteria. bvFTD may be attributed to frontotemporal lobar degeneration (FTLD)-tau, FTLD-TDP, and FTLD-FUS. Some language endophenotype FTLD were included in bvFTD (n = 3). The CBS PSP continuum included patients with CBS clinical phenotypes that evolved into PSP during follow-up.

CSF collection: CSF from individuals with AD and AMC in the WashU-A cohort were collected via a catheter as previously described. CSF from AMC and individuals with symptomatic AD, PSP, CBS, and bvFTD in the WashU-B cohort were obtained via LP with gravity collection and centrifugation as previously described. CSF from MAPT mutation families was collected according to the standardized protocol at the Biomarker Core at the Washington University School of Medicine. CSF from individuals with brain tumors was obtained via lumbar drain using a catheter before or after surgery.

CSF from the Montpellier cohort was collected using the standardized protocol for the collection, centrifugation, and storage at Memory Resources and Research Center of Montpellier. Briefly, the atraumatic needle was used for LP, with CSF collected into 10 mL polypropylene tube and protein low binding Eppendorf tubes. CSF was not centrifuged before aliquoting and storage at -80° C. CSF tau and pT181 concentrations were measured using the standardized commercially available INNOTEST sandwich ELISA X-MAP following Fujirebio instructions. CSF Aβ 42 and Aβ 40 were measured using INNOTEST sandwich ELISA from Fujirebio.

Sequential IP and MS methods for CSF Aβ and Tau: CSF Aβ was analyzed as previously described with the following modifications. Master mix containing detergent and chaotropic reagents (final 1% NP-40, 5 mmol/L guanidine, protease inhibitor cocktail) and internal standards for tau (¹⁵N labeled 2N4R recombinant tau) and Aβ (¹⁵N labeled synthetic Aβ 40, and 42) were prepared in low-binding Axygen tubes (Fisher Scientific, Pittsburgh, PA, USA, MCT-175). 500-1000 µL of CSF was added and immunoprecipitated with the HJ5.1 mid-domain Aβ antibody. After washing, samples were digested with LysN protease, desalted, and analyzed by Xevo TQ-S mass spectrometer (Waters Corporation, Milford, MA, USA).

CSF tau and ptau were analyzed as previously described with the following modifications. Post-HJ5.1 immunoprecipitated CSF samples containing tau internal standards were sequentially immunoprecipitated with Tau1 mid-domain and HJ8.5 N-terminus tau antibodies. After washing, samples were digested with trypsin, desalted, and analyzed on Orbitrap Fusion mass spectrometer (Thermo Fisher Scientific, San Jose, CA, USA). MS method measuring pT217 and pT181 was described previously.

Amyloid and Tau PET imaging: Amyloid PiB-PET, AV45-PET, and Tau AV1451-PET imaging measurements were performed in a subset of AD and AMC participants from the Knight ADRC at Washington University School of Medicine. Data were collected and processed as previously described.

### Example 2 - pTau Comparison in Non-AD Tauopathies: LOAD vs UCSF vs NCRAD vs CTRL vs Tangles

Phosphorylation on T217 increases with amyloid-beta as previously reported. Abnormal tau phosphorylation was defined as values above the cutoff defined by samples from participants in the groups: amyloid beta negative (AB-), cognitively normal (CN), young normal controls (YNC), brain metastasis (BM) or control (CTRL)).

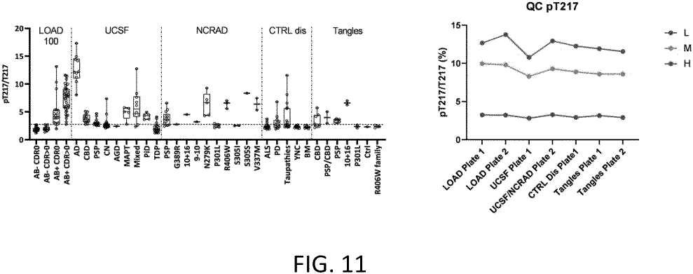

Considering this criteria, abnormal tau phosphorylation was observed in a large proportion of participants with corticobasal degeneration (CBD), Progressive supranuclear palsy (PSP), Pick Disease (PiD). This trend is observed for CBD and PSP in 3 independent cohorts (UCSF, NCRAD, Tangles). The values observed are in the same range as amyloid positive participants without clinical symptoms (AB+ CDR0). Also, MAPT mutations N279K, S305S, V337M, 10+16 seem to induce abnormal CSF ptau217. R406W as shown above has abnormal ptau 217 as well. Thus, an increase of ptau217 together with normal CSF Abeta 42/40 appears to be useful to identify participants at risk of non-AD tauopathy (FIG. 11).

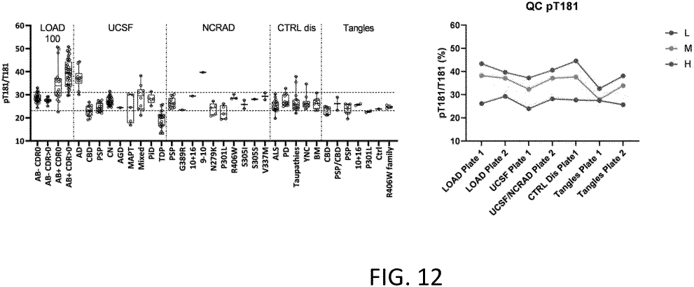

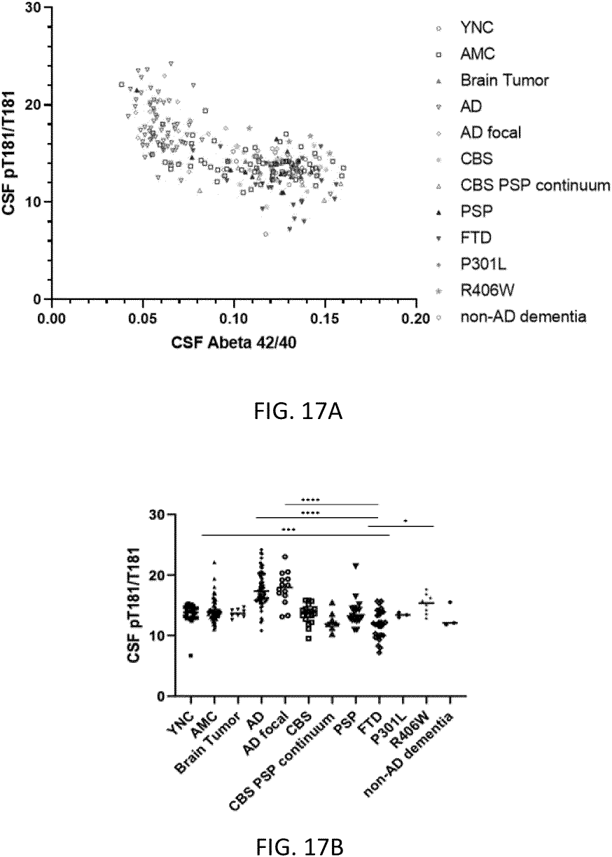

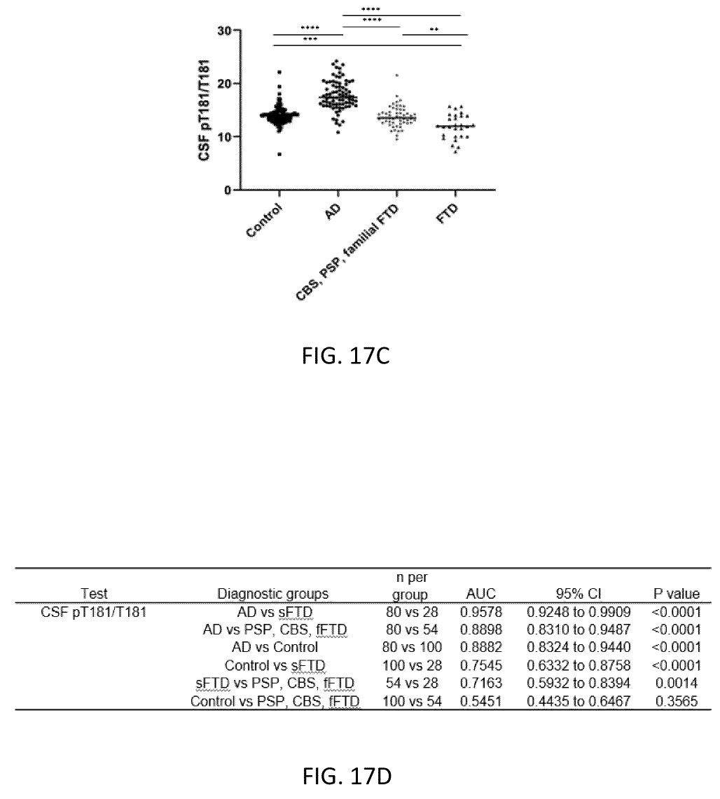

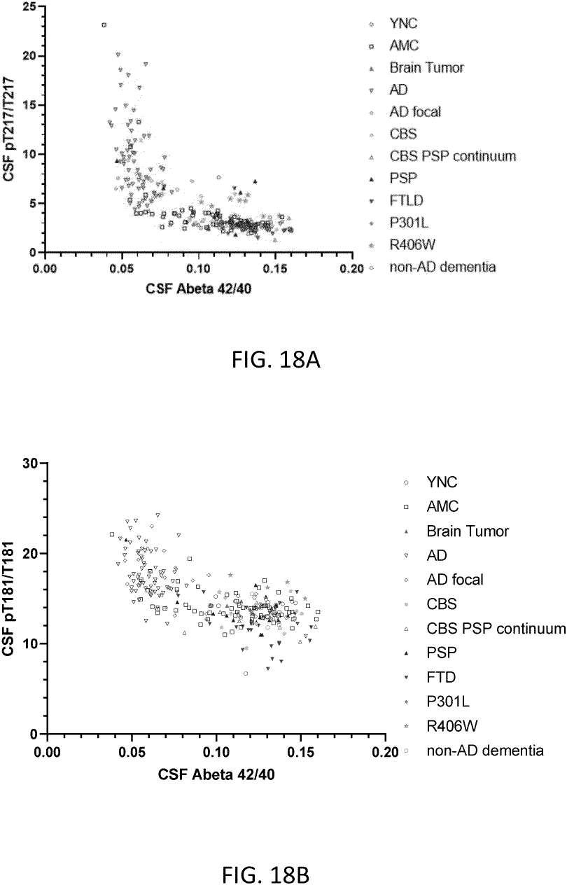

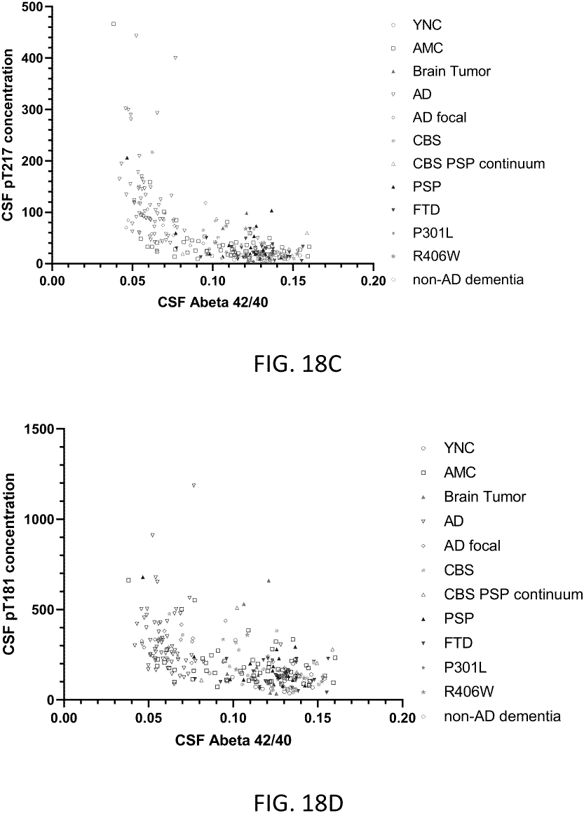

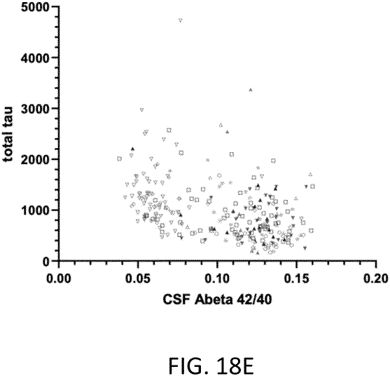

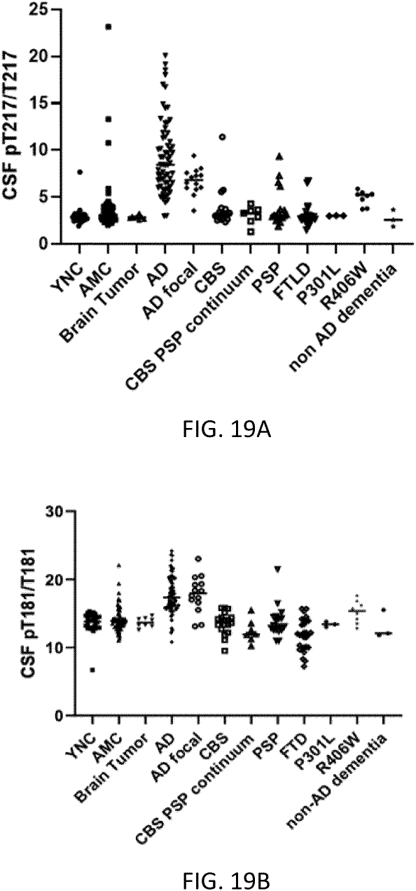

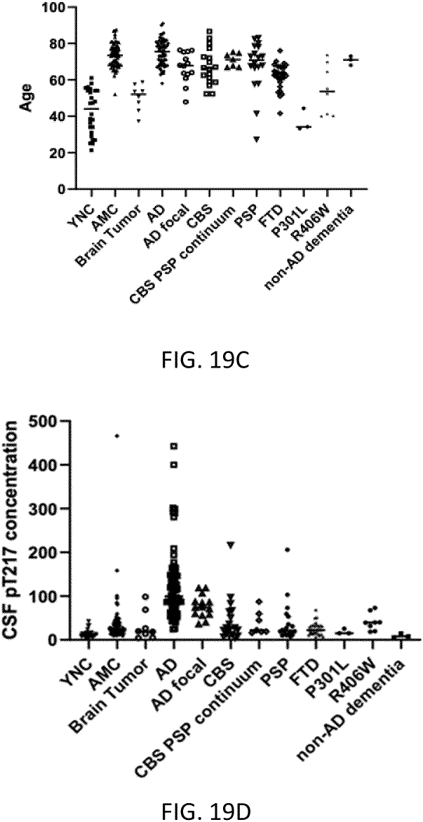

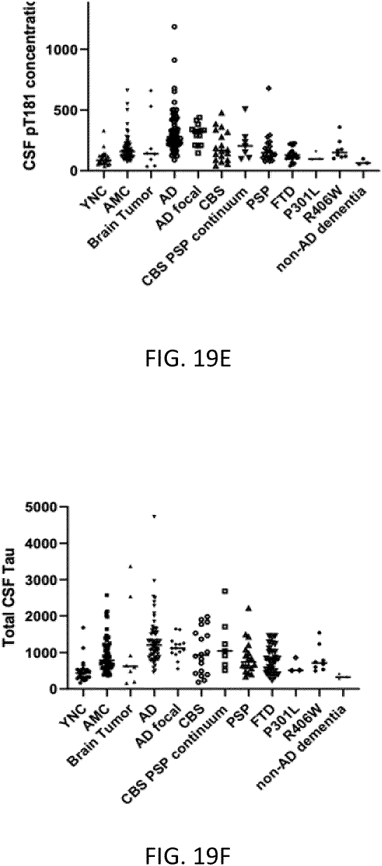

FIG. 12 shows ptau181 is not sensitive enough to detect change of tau phosphorylation in non-AD tauopathies. However, lower ptau181 was observed in TDP samples. Moreover, as shown in FIGS. 17-19, sporadic FTD (including FTD-tau, FTD-FUS, FTD-TDP43) have decreased CSF pT181/T181 ratio. Thus, pT181/T181 can separate sporadic FTD from other tauopathies when ratio is measured.

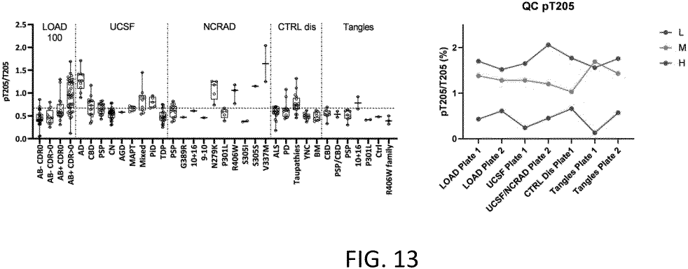

As shown in FIG. 13, ptau205 is increased mainly in amyloid positive symptomatic (AB+ CDR>0) and AD. N279K, S305S, V337M, and R406W but not 10+16 are abnormally phosphorylated as found for ptau217. Some non-AD tauopathies (CBD, PSP and Pick disease) might be slightly higher than controls groups but this site would be likely less sensitive than ptau217.

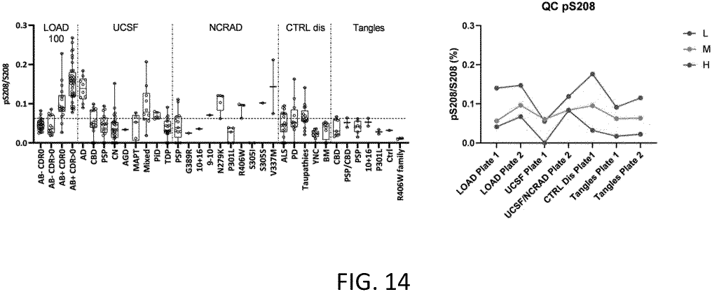

pTau208 associates with ptau217 in LOAD cohort but appears to be relatively lower in AD compared to what was observed for ptau217. N279K, S305S, V337M and R406W but not 10+ 16 are abnormally phosphorylated. Some non-AD tauopathies (CBD, PSP and Pick disease) might be slightly higher than controls groups but this site would be likely less sensitive than ptau217 (FIG. 14).

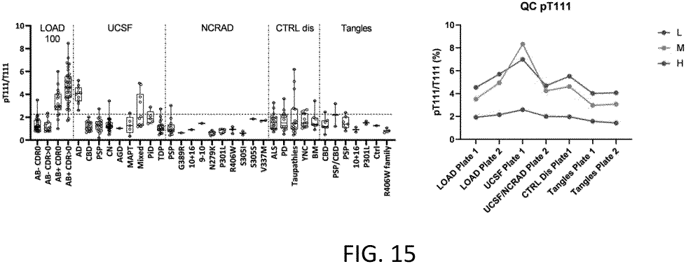

FIG. 15 shows ptau111 associates with ptau217 in LOAD cohort but appears to be relatively lower in AD compared to what was observed for ptau217. No abnormal phosphorylation is observed for non-AD tauopathies as CBD, PSP or MAPT mutation found hyperphosphorylated for ptau217. This could suggest ptau111 is truly AD specific and could be used to differentiate AD from non-AD tauopathies being abnormally phosphorylated for ptau217.

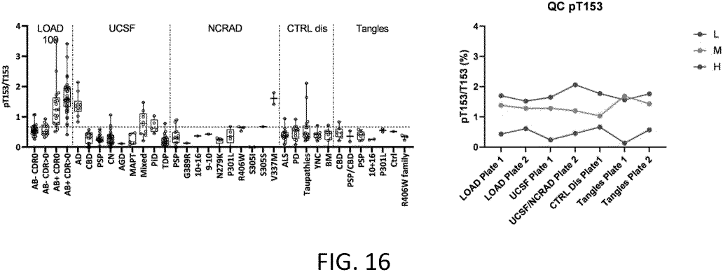

As shown in FIG. 16, as with ptau111, ptau153 associates with ptau217 in LOAD cohort but appears to be relatively lower in AD compared to what was observed for ptau217. No abnormal phosphorylation is observed for non-AD tauopathies as CBD, PSP or MAPT mutation found hyperphosphorylated for ptau217. This suggests ptau153 is truly AD specific and could be used to differentiate AD from non-AD tauopathies being abnormally phosphorylated for ptau217.

What is claimed is:

1-49. (canceled)

50. A method to diagnose a subject having a symptom of Alzheimer’s disease, the method comprising:
(a) providing a processed CSF or blood sample obtained from the subject, wherein the CSF or blood sample is enriched for one or more phospho-tau and Aβ species; and
(b) diagnosing the subject as having, or at an increased risk of having, AD or a non-AD tauopathy when a normal or significantly different Aβ 42/40 value is detected and a significantly different phospho-tau value is detected relative to a phospho-tau value of the same species and an Aβ 42/40 value in a healthy control population or disease population.

51. The method of claim 50, further comprising quantifying, in the processed sample, a pT217/T217 value and an Aβ 42/40 value.

52. The method of claim 51, wherein the subject is diagnosed as having, or at an increased risk of having, a non-AD tauopathy when a normal Aβ 42/40 value is detected and an increased pT217/T217 value is detected relative to a pT217/T217 value of the and an Aβ 42/40 value in a healthy control population.

53. The method of claim 51, wherein the subject is diagnosed as having, or at an increased risk of having, a non-AD tauopathy when an increased Aβ 42/40 value is detected and a decreased pT217/T217 value is detected relative to a pT217/T217 value of the and an Aβ 42/40 value in an AD population.

54. The method of claim 51, further comprising calculating determining a composite pT217/T217 x Aβ 42/40 value, wherein an increased composite value relative to a healthy control population indicates the subject as having or at an increased risk for AD or a non-AD tauopathy.

55. The method of claim 54, wherein the subject is diagnosed as having or at an increased risk of having FTD when an increased composite value relative to a healthy control population and an increased composite value relative to an AD population.

56. The method of claim 50, further comprising quantifying, in the processed sample, a pT181/T181 value.

57. The method of claim 56, wherein the subject is diagnosed as having or at an increased risk of having FTD when a decreased pT181/T181 value is detected relative to a healthy control population.

58. The method of claim 56, wherein the subject is excluded from an AD diagnosis when the detected pT181/T181 value is decreased relative to an AD population.

59. The method of claim 50, further comprising quantifying, in the processed sample, a pT153/T153 value.

60. The method of claim 59, wherein the subject is diagnosed as having or at an increased risk of having AD when the detected pT153/T153 value is increased relative to a healthy control population and/or a non-AD tauopathy population.

61. The method of claim 59, wherein the subject is diagnosed as having, or at an increased risk of having, a non-AD tauopathy when a normal Aβ 42/40 value is detected and an increased pT153/T153 value is detected relative to a pT153/T153 value of the and an Aβ 42/40 value in a healthy control population.

62. The method of claim 50, further comprising quantifying, in the processed sample, a pT111/T111 value.

63. The method of claim 62, wherein the subject is diagnosed as having or at an increased risk of having AD when the detected pT111/T111 value is increased relative to a healthy control population and/or a non-AD tauopathy population.

64. The method of claim 62, wherein the subject is diagnosed as having, or at an increased risk of having, a non-AD tauopathy when a normal Aβ 42/40 value is detected and an increased pT111/T111 value is detected relative to a pT111/T111 value of the and an Aβ 42/40 value in a healthy control population.

65. The method of claim 50, further comprising quantifying, in the processed sample, a pT205/T205 value.

66. The method of claim 65, wherein the subject is diagnosed as having or at an increased risk of having a tauopathy when the detected pT205/T205 value is increased relative to a healthy control population.

67. The method of claim 50, further comprising quantifying, in the processed sample, a pS208/S208 value.

68. The method of claim 67, wherein the subject is diagnosed as having or at an increased risk of having a tauopathy when the detected pS208/S208 value is increased relative to a healthy control population.

69. A method of discriminating a tauopathy, the method comprising
(a) providing a processed CSF or blood sample obtained from a subject, wherein the CSF or blood sample is enriched for one or more phospho-tau and Aβ species; and
(b) quantifying, in the processed sample, a phospho-tau value and an Aβ 42/40 value, wherein a significantly different phospho-tau value and/or Aβ 42/40 value discriminates a tauopathy from a healthy state.

70. The method of claim 69, further comprising quantifying, in the processed sample, a pT217/T217 value and an Aβ 42/40 value.

71. The method of claim 70, wherein the subject is diagnosed as having, or at an increased risk of having, a non-AD tauopathy when a normal Aβ 42/40 value is detected and an increased pT217/T217 value is detected relative to a pT217/T217 value of the and an Aβ 42/40 value in a healthy control population.

72. The method of claim 70, wherein the subject is diagnosed as having, or at an increased risk of having, a non-AD tauopathy when an increased Aβ 42/40 value is detected and a decreased pT217/T217 value is detected relative to a pT217/T217 value of the and an Aβ 42/40 value in an AD population.

73. The method of claim 70, further comprising calculating determining a composite pT217/T217 x Aβ 42/40 value, wherein an increased composite value relative to a healthy control population indicates the subject as having or at an increased risk for AD or a non-AD tauopathy.

74. The method of claim 73, wherein the subject is diagnosed as having or at an increased risk of having FTD when an increased composite value relative to a healthy control population and an increased composite value relative to an AD population.

75. The method of claim 69, further comprising quantifying, in the processed sample, a pT181/T181 value.

76. The method of claim 75, wherein the subject is diagnosed as having or at an increased risk of having FTD when a decreased pT181/T181 value is detected relative to a healthy control population.

77. The method of claim 75, wherein the subject is excluded from an AD diagnosis when the detected pT181/T181 value is decreased relative to an AD population.

78. The method of claim 69, further comprising quantifying, in the processed sample, a pT153/T153 value.

79. The method of claim 78, wherein the subject is diagnosed as having or at an increased risk of having AD when the detected pT153/T153 value is increased relative to a healthy control population and/or a non-AD tauopathy population.

80. The method of claim 78, wherein the subject is diagnosed as having, or at an increased risk of having, a non-AD tauopathy when a normal Aβ 42/40 value is detected and an increased pT153/T153 value is detected relative to a pT153/T153 value of the and an Aβ 42/40 value in a healthy control population.

81. The method of claim 69, further comprising quantifying, in the processed sample, a pT111/T111 value.

82. The method of claim 81, wherein the subject is diagnosed as having or at an increased risk of having AD when the detected pT111/T111 value is increased relative to a healthy control population and/or a non-AD tauopathy population.

83. The method of claim 81, wherein the subject is diagnosed as having, or at an increased risk of having, a non-AD tauopathy when a normal Aβ 42/40 value is detected and an increased pT111/T111 value is detected relative to a pT111/T111 value of the and an Aβ 42/40 value in a healthy control population.

84. The method of claim 69, further comprising quantifying, in the processed sample, a pT205/T205 value.

85. The method of claim 84, wherein the subject is diagnosed as having or at an increased risk of having a tauopathy when the detected pT205/T205 value is increased relative to a healthy control population.

86. The method of claim 69, further comprising quantifying, in the processed sample, a pS208/S208 value.

87. The method of claim 86, wherein the subject is diagnosed as having or at an increased risk of having a tauopathy when the detected pS208/S208 value is increased relative to a healthy control population.

88. A method for treating a subject in need thereof, the method comprising
(a) providing a processed CSF or blood sample obtained from a subject, wherein the CSF or blood sample is enriched for one or more phospho-tau and Aβ species and quantifying, in the processed sample, a phospho-tau value and an Aβ 42/40 value; and
(b) administering a pharmaceutical composition to the subject when tauopathy is detected relative to a healthy state.

89. The method of claim 88, further comprising quantifying, in the processed sample, a pT217/T217 value and an Aβ 42/40 value.

90. The method of claim 89, wherein the subject is treated for a non-AD tauopathy when a normal Aβ 42/40 value is detected and an increased pT217/T217 value is detected relative to a pT217/T217 value of the and an Aβ 42/40 value in a healthy control population.

91. The method of claim 89, wherein the subject is treated for a non-AD tauopathy when an increased Aβ 42/40 value is detected and a decreased pT217/T217 value is detected relative to a pT217/T217 value of the and an Aβ 42/40 value in an AD population.

92. The method of claim 90, further comprising calculating determining a composite pT217/T217 x Aβ 42/40 value, wherein the subject is treated when an increased composite value relative to a healthy control population.

93. The method of claim 91, wherein the subject is the subject is treated for FTD when an increased composite value relative to a healthy control population and an increased composite value relative to an AD population.

94. The method of claim 88, further comprising quantifying, in the processed sample, a pT181/T181 value.

95. The method of claim 94, wherein the subject is the subject is treated for FTD when a decreased pT181/T181 value is detected relative to a healthy control population.

96. The method of claim 94, wherein the subject is with a anti-tau therapeutic when the detected pT181/T181 value is decreased relative to an AD population.

97. The method of claim 88, further comprising quantifying, in the processed sample, a pT153/T153 value.

98. The method of claim 97, wherein the subject is the subject is treated when the detected pT153/T153 value is increased relative to a healthy control population and/or a non-AD tauopathy population.

99. The method of claim 97, wherein the subject is the subject is treated when a normal Aβ 42/40 value is detected and an increased pT153/T153 value is detected relative to a pT153/T153 value of the and an Aβ 42/40 value in a healthy control population.

100. The method of claim 88, further comprising quantifying, in the processed sample, a pT111/T111 value.

101. The method of claim 100, wherein the subject is the subject is treated for AD when the detected pT111/T111 value is increased relative to a healthy control population and/or a non-AD tauopathy population.

102. The method of claim 100, wherein the subject is the subject is treated for a non-AD tauopathy when a normal Aβ 42/40 value is detected and an increased pT111/T111 value is detected relative to a pT111/T111 value of the and an Aβ 42/40 value in a healthy control population.

103. The method of claim 88, further comprising quantifying, in the processed sample, a pT205/T205 value.

104. The method of claim 103, wherein the subject is the subject is treated for a tauopathy when the detected pT205/T205 value is increased relative to a healthy control population.

105. The method of claim 88, further comprising quantifying, in the processed sample, a pS208/S208 value.

106. The method of claim 105, wherein the subject is the subject is treated for a tauopathy when the detected pS208/S208 value is increased relative to a healthy control population.

107. A method for selecting a subject in a clinical trial, the method comprising
(a) providing a processed CSF or blood sample obtained from a subject, wherein the CSF or blood sample is enriched for one or more phospho-tau and Aβ species;
(b) quantifying, in the processed sample, a phospho-tau value and Aβ 42/40 value; and
(c) selecting the subject into a clinical trial for AD or a non-AD tauopathy based on the quantified a phospho-tau value and Aβ 42/40 value.

108. The method of claim 107, further comprising quantifying, in the processed sample, a pT217/T217 value and an Aβ 42/40 value.

109. The method of claim 108, wherein the subject is selected for the non-AD tauopathy clinical or excluded from the AD clinical trial when a normal Aβ 42/40 value is detected and an increased pT217/T217 value is detected relative to a pT217/T217 value of the and an Aβ 42/40 value in a healthy control population.

110. The method of claim 108, wherein the subject is selected for a non-AD tauopathy clinical trial or excluded from an AD clinical trial when an increased Aβ 42/40 value is detected and a decreased pT217/T217 value is detected relative to a pT217/T217 value of the and an Aβ 42/40 value in an AD population.

111. The method of claim 109, further comprising calculating determining a composite pT217/T217 x Aβ 42/40 value, wherein the subject is selected in the treatment arm of the clinical trial when an increased composite value relative to a healthy control population.

112. The method of claim 109, wherein the subject is the subject is selected for a non-AD tauopathy clinical trial for FTD or excluded from an AD clinical trial when an increased composite value relative to a healthy control population and an increased composite value relative to an AD population.

113. The method of claim 107, further comprising quantifying, in the processed sample, a pT181/T181 value.

114. The method of claim 113, wherein the subject is selected for a non-AD tauopathy clinical trial for FTD or excluded from an AD clinical trial when a decreased pT181/T181 value is detected relative to a healthy control population.

115. The method of claim 113, wherein the subject is selected for a non-AD clinical trial or excluded from an AD clinical trial when the detected pT181/T181 value is decreased relative to an AD population.

116. The method of claim 107, further comprising quantifying, in the processed sample, a pT153/T153 value.

117. The method of claim 116, wherein the subject is the subject is selected for the treatment arm of a clinical trial when the detected pT153/T153 value is increased relative to a healthy control population and/or a non-AD tauopathy population.

118. The method of claim 116, wherein the subject is the subject is selected for the treatment arm of a clinical trial when when a normal Aβ 42/40 value is detected and an increased pT153/T153 value is detected relative to a pT153/T153 value of the and an Aβ 42/40 value in a healthy control population.

119. The method of claim 107, further comprising quantifying, in the processed sample, a pT111/T111 value.

120. The method of claim 119, wherein the subject is the subject is selected for an AD clinical trial or excluded for a non-AD tauopathy clinical trial when the detected pT111/T111 value is increased relative to a healthy control population and/or a non-AD tauopathy population.

121. The method of claim 119, wherein the subject is the subject is selected for a non-AD tauopathy clinical trial or excluded from an AD clinical trial when a normal Aβ 42/40 value is detected and an increased pT111/T111 value is detected relative to a pT111/T111 value of the and an Aβ 42/40 value in a healthy control population.

122. The method of claim 107, further comprising quantifying, in the processed sample, a pT205/T205 value.

123. The method of claim 122, wherein the subject is the subject is selected for the treatment arm of a clinical trial when the detected pT205/T205 value is increased relative to a healthy control population.

124. The method of claim 107, further comprising quantifying, in the processed sample, a pS208/S208 value.

125. The method of claim 124, wherein the subject is the subject is selected for the treatment arm of a clinical trial when the detected pS208/S208 value is increased relative to a healthy control population.

126. A method for measuring disease progression of a tauopathy in a subject, the method comprising:
(a) providing a first processed CSF or blood sample and a second processed CSF or blood sample, wherein each processed sample is obtained from the subject, and wherein each CSF or blood sample is enriched for one or more phospho-tau and Aβ species;
(b) quantifying, in the processed sample, a phospho-tau value and Aβ 42/40 value; and
(c) calculating the difference between the quantified phospho-tau species and Aβ 42/40 value in the second sample and the first sample, wherein a statistically significant change in the quantified phospho-tau species and/or Aβ 42/40 value in the second sample indicates progression of the subject’s disease.

127. The method of claim 126, further comprising quantifying, in the processed sample, a pT217/T217 value and an Aβ 42/40 value.

128. The method of claim 127, wherein disease progression is indicated when a normal Aβ 42/40 value is detected and an increased pT217/T217 value is detected.

129. The method of claim 127, wherein disease progression is indicated when an increased Aβ 42/40 value is detected and a decreased pT217/T217 value is detected.

130. The method of claim 127, further comprising calculating a composite pT217/T217 x Aβ 42/40 value, wherein an increased composite value in the second sample indicates progression of the subject’s disease.

131. The method of claim 127, further comprising quantifying, in the processed sample, a pT181/T181 value.

132. The method of claim 131, wherein disease progression is indicated when a decreased pT181/T181 value is detected.

133. The method of claim 126, further comprising quantifying, in the processed sample, a pT153/T153 value.

134. The method of claim 133, wherein disease progression is indicated when the detected pT153/T153 value is increased.

135. The method of claim 133, wherein disease progression of a non-AD tauopathy is indicated when a normal Aβ 42/40 value is detected and an increased pT153/T153 value is detected.

136. The method of claim 126, further comprising quantifying, in the processed sample, a pT111/T111 value.

137. The method of claim 136, wherein disease progression is indicated when the detected pT111/T111 value is increased.

138. The method of claim 136, wherein disease progression of a non-AD tauopathy is indicated when a normal Aβ 42/40 value is detected and an increased pT111/T111 value is detected.

139. The method of claim 126, further comprising quantifying, in the processed sample, a pT205/T205 value.

140. The method of claim 139, wherein disease progression is indicated when the detected pT205/T205 value is increased.

141. The method of claim 126, further comprising quantifying, in the processed sample, a pS208/S208 value.

142. The method of claim 141, wherein disease progression of a tauopathy is indicated when the detected pS208/S208 value is increased.

143. A kit for isolating and measuring tau and/or Aβ, the kit comprising at least one isotope labeled internal standard, an antibody that has affinity for one or more tau isoforms and/or an antibody that has affinity for one or more Aβ isoforms.
